(19) United States
(12) Patent Application Publication
(10) Pub. No.: US 2014/0087964 A1
(43) Pub. Date: Mar. 27, 2014

(54) COMPOSITIONS AND METHODS FOR DETECTING ABERRANT REGULATION, EXPRESSION, AND LEVELS OF HGH

(71) Applicants: David E. Bruns, Greenwood, VA (US); Doris M. Haverstick, Charlottesville, VA (US); Brian N. Kelly, Holladay, UT (US)

(72) Inventors: David E. Bruns, Greenwood, VA (US); Doris M. Haverstick, Charlottesville, VA (US); Brian N. Kelly, Holladay, UT (US)

(73) Assignee: UNIVERSITY OF VIRGINIA PATENT FOUNDATION, Charlottesville, VA (US)

(21) Appl. No.: 14/035,422

(22) Filed: Sep. 24, 2013

Related U.S. Application Data
(60) Provisional application No. 61/704,716, filed on Sep. 24, 2012.
(60) Provisional application No. 61/704,707, filed on Sep. 24, 2012.

(51) Int. Cl. C12Q 1/68

(52) U.S. Cl. CPC C12Q 1/6876
USPC 506/9; 435/6.12

(57) ABSTRACT

Detection of human growth hormone use is very challenging due to the short half-life of circulating human growth hormone and the fact that the recombinant human growth hormone used is identical in protein sequence as the native, naturally produced growth hormone. The chief objective of this invention is to discover a marker(s) that will identify use of recombinant human growth hormone use. In the case of this invention, we have discovered specific circulating micro RNA (miRNA) that can be detected and quantified in plasma of individuals taking recombinant human growth hormone.

### CROSS REFERENCE TO RELATED APPLICATIONS

This application is entitled to priority pursuant to 35 U.S.C. §119(e) to U.S. provisional patent application Nos. 61/704,707, filed on Sep. 24, 2012 and 61/704,716, filed on Sep. 24, 2012. The entire disclosures of the afore-mentioned patent applications are incorporated herein by reference.

### BACKGROUND

Nucleic acids in blood and body fluids are an increasingly important class of analytes in clinical medicine and are attractive targets for measurement in anti-doping efforts. In medicine, nucleic acids in circulating cells have been sequenced and quantified to characterize the expression patterns of genes in leukemic blood cells and to detect the presence of circulating tumor cells from solid tissues. The presence of “naked” circulating DNA in human plasma was described in 1948, but its diagnostic potential was barely recognized for the next three decades. In recent years, measurements of specific DNA sequences in plasma have shown utility in many areas of medicine, ranging from prediction of spread of cancer to detection of liver disease. Because fetal DNA is present in the mother's blood, its measurement is now used to determine the sex of the fetus early in gestation and to identify the presence of a range of fetal conditions, including Down syndrome. Plasma contains not only DNA but also a broad range of RNAs including messenger RNA, microRNA, and other small RNAs that have yet to be characterized in detail. Both DNA and RNA are present in other body fluids including urine and saliva, and measurements of them are already showing diagnostic utility.

Nucleic acid testing has appealing aspects for doping control: (1) DNA and many RNAs are quite stable in blood and body fluids as is prerequisite for testing that may involve sample collection at geographically remote locations and may require repeated analysis of samples that must be stored for years; (2) the technology to measure nucleic acid sequences is widely used in clinical and research laboratories, and, importantly the technology is advancing dramatically. The cost of testing is plummeting and the speeds are increasing for both sequencing and quantification of nucleic acids; and (3) the study of the roles of some nucleic acids, currently in early stages, is already demonstrating unanticipated functions such as the roles of microRNAs in regulation of cellular growth.

The Hypothalamus-Pituitary-IGF-I Axis and Doping with Growth Hormone

Recombinant human GH (rhGH) increases lipolytic activity, has anabolic properties, and is difficult to detect, making it an attractive substance for athletes looking to gain an advantage over their competition. Detection of abuse of rhGH may be aided by exploring the effects of hGH on nucleic acids (DNA and RNAs) in blood cells and plasma and, potentially, in other body fluids.

hGH is produced in, and released into circulation, from the anterior pituitary. Once in circulation, hGH targets a number of tissues, including the liver and lymphocytes. hGH stimulates the liver to produce insulin-like growth factor 1 (IGF-1), which mediates the effects of hGH on additional tissues. These growth effects require increased protein synthesis. In contrast, IGF-1 and hGH act as negative regulators of synthesis of hGH. Thus, in this negative feedback system, both the hormone, hGH, and its effector, IGF-I, inhibit further production and release of the hormone, hGH. A central hypothesis of our research is that treatment with rhGH (or substances that mimic hGH or increase its release from pituitary) alters the level of the hGH messenger RNA (mRNA) and that this is detectable in cells and fluids outside the pituitary. Preliminary studies of Thakkar, Butt, Powrie, Holt, and Swaminathan have suggested that hGH mRNA is increased in blood from patients with hGH-secreting tumors [4].

### MicroRNAs and Growth Hormone

A second effect of hormone administration on nucleic acids is changes in the expression pattern of a newly appreciated class of RNA called microRNA (miRNA). Emerging data show that miRNAs have diagnostic potential in cancer. Recently, a group from the Swiss Laboratory for Doping Analysis discovered an increase in miR-144 concentration in plasma of individuals who have taken a continuous erythropoietin receptor activating (CERA) hormone. This increase in miR-144 concentration lasted for 4 weeks after injection of the hormone, providing evidence that miRNA may prove useful in doping control.

Athletes illicitly use human growth hormone to enhance their performance in competition. As such, the use of human growth hormone has been banned by the World Anti-Doping Agency (WADA) and the United States Anti-Doping Agency (USADA). Current methods of detecting use of human growth hormone involve assays that differentiate between the separate isoforms of human growth hormone in circulation. Pharmaceutical companies produce recombinant human growth hormone as a single, 22-kilo-dalton isoform, while in circulation, there are 5 different isoforms, including a 20-kilo-dalton form. If the “iso-form ratio” (22 kDa/20 kDa) is greater than the allowed maximum, an adverse analytical finding (presumptive doping offense) is filed with the appropriate sporting authorities. The major problem with this current assay is that within 24 hours of administration, this form of evidence for hGH use is absent. In addition, the 20-kDa form has also been synthesized and thus athletes can now inject both forms to keep the isoform ratio below the allowed maximum. Thus, a need remains for an improved assay to catch abuse and deter athletes from using rhGH. Moreover, existing technologies cannot distinguish normal growth hormone made by the body from growth hormone provided by a pharmaceutical company. A recently proposed approach is based on measuring the concentrations, in serum, of proteins that are increased by growth hormone in bone and liver. This approach is unproven and considerable work remains to be done to see if it is viable.

There is a long-felt need in the art for compositions and methods useful for detecting and diagnosing diseases and disorders associated with aberrant GHRH and hGH expression and levels and with changes in expression and levels of GHRH and hGH associated with the exogenous administration of GH or other substances. There is also a long-felt need for new methods and biomarkers to monitor changes after growth hormone administration to determine if growth hormone has been administered, if it is working properly, or if it being used at the proper dosages. The present invention satisfies these needs.

### SUMMARY OF THE INVENTION

The present application discloses that growth hormone administration can regulate levels of microRNAs and that circulating growth hormone mRNA can be detected at low levels using a new and sensitive assay disclosed herein. Changes in mRNA levels are associated with pathologies of the GHRH and hGH pathways/axis as well as with administration of exogenous regulators or substances that regulate the pathway or are part of the pathway.

The present application further discloses that administration of growth hormone or agents that mimic hGH or increase its secretion by the pituitary, causes a change in levels of specific miRNAs. Therefore, these miRNAs are useful as biomarkers for changes in growth hormone levels, particularly when exogenous growth hormone or biologically active fragments, hormones, or derivatives thereof are administered to a subject. These miRNAs appear to be regulated by administration of growth hormone or biologically active fragments, hormones, or derivatives thereof.

In one embodiment, the present invention provides biomarkers for detecting the use of exogenous growth hormone. In one aspect, the biomarkers are miRNAs. In one aspect, the present invention provides compositions and methods for detecting and measuring the levels of the miRNAs and for monitoring the treatment of subjects requiring therapy with growth hormone or agents that mimic hGH or increase its secretion by the pituitary (GH agonists). In one aspect, the present invention provides compositions and methods for detecting and measuring the levels of the miRNAs and for monitoring those miRNAs in athletes.

In one embodiment, the present invention encompasses detection of certain miRNAs in circulation (blood) from individuals receiving human growth hormone, or agonists of growth hormone activity or of its signalling pathway, administered as part of a growth hormone replacement therapy regimen. In one aspect, the miRNAs that are specifically found only in individuals taking recombinant human growth hormone are: hsa-miR-510, hsa-miR-200b*, and hsa-miR-1272. In one aspect, these markers are detected using microarray. These three biomarkers can be used as biomarkers individually or in combination with one another or other biomarkers, including other miRNAs. The presence of one or more of these biomarkers is an indication that the subject has taken a biologically active growth hormone or a biologically active fragment, homolog, derivative, or analog thereof.

In one embodiment, the invention encompasses detecting and measuring changes in levels of miRNAs in subjects who take growth hormone or fragments, homologs, or derivatives thereof. In one aspect, administration of growth hormone, or agonists of growth hormone activity or its signaling pathways, causes a decrease in the levels of some miRNAs. In one aspect, the miRNAs decreasing upon growth hormone treatment are selected from the group consisting of miR-663 (NCBI Ref. Seq. No. NR_{—}030363.1), miR-2861 (NCBI Ref. Seq. No. NR_{—}036055.1), miR-3185 (NCBI Ref. Seq. No. NR_{—}036150.1), and miR-3152 (NCBI Ref. Seq. No. NR_{—}036107.1). In one aspect, samples are pre-amplified, and in another aspect, the samples are not preamplified.

In one aspect, circulating miRNAs that change levels when growth hormone levels change are useful as a biomarker for the diseases, disorders, and conditions described herein as well as for determining whether exogenous sources of growth hormone or growth hormone regulators have been administered to a subject.

The invention encompasses the use of growth hormone for treatment and as a means to regulate miRNAs sensitive to exogenous growth hormone. In one aspect, the growth hormone is human growth hormone. In another aspect, it is recombinant growth hormone. Biologically active fragments, homologs, and derivatives are also useful in the practice of the invention.

It will also be appreciated that based on the discovery that four miRNAs decrease upon growth hormone administration, that growth hormone can be administered to a subject with the intent to decreased the levels of the four miRNAs that are sensitive to growth hormone administration. The amount of growth hormone, or fragments, homologs, or derivatives thereof that is administered can be based on the type of disease or disorder being treated and on the age, sex, health, and weight of the individual. One of ordinary skill in the art will appreciated that the growth hormone can be, for example, purified, recombinant, or synthetic. Mimics or antagonists or growth hormone or its activity or signaling pathway, include, for example, GHRH and its mimics and agonists.

An advantage over existing alternative products includes the ability to use a marker produced by the natural physiology of the individual taking the recombinant human growth hormone rather than looking for the hormone itself in circulation. This provides a distinct advantage in that the “window of detection” may be significantly longer than with the current method.

Testing of athletes for use of growth hormone is likely to increase in the near future. The National Football League owners and players have reached agreement to begin testing. Other organizations are likely to follow. At present, the only alternative is to measure the two major forms of growth hormone in blood. This approach to detection relies on the assumption that the user will inject only one of the two forms, making the ratio of the two forms abnormal. This approach is limited by the fact that, now, both forms of growth hormone can be injected. Moreover, the injected growth hormone disappears from the blood rapidly (hours) after injection and thus the ratio returns to normal. A recently proposed approach is based on measuring the concentrations, in serum, of proteins that are increased by growth hormone in bone and liver, but the present approach is entirely different and unrelated to proteins.

The present invention also provides a new and more sensitive assay for measuring GH mRNA levels. The availability of reagents to detect expression and levels of GH (protein or mRNA) or miRNA levels regulated by GH makes application of the invention feasible and immediate. In one aspect, the present application discloses compositions and methods useful to detect use of exogenous growth hormone and/or use of agents that increase growth hormone and growth hormone activity in a subject. In one aspect, a change in the levels of circulating GH mRNA is an indication that a subject has taken an exogenous growth hormone or a biologically fragment, homolog, analog, or derivative thereof. In one aspect, the subject is a human. In another aspect, the subject is an athlete.

The methods of the invention are also useful for monitoring changes in GH mRNA over time in a subject and correlating that change with GH levels or the presence of exogenous hGH or regulators of hGH.

In one aspect, the GH mRNA assay includes the use of nested PCR.

The assay has been optimized in order to detect and measure very low quantities of GH mRNA, relative to known assays. The assay encompasses measuring the amount of growth hormone mRNA in a sample by first purifying RNA from the sample. One of ordinary skill in the art will appreciate that more than one method can be used for purification. In one embodiment, the purified RNA is then subjected to reverse transcription and a first round of PCR using forward and reverse primers for growth hormone mRNA. Alternatively, the purified RNA is amplified using another method, such as enzymatic amplification. In one aspect, the primers are exon-exon junction spanning primers. PCR products are obtained and can be quantified. One of ordinary skill in the art will appreciate that various methods can be used to quantify the PCR products and that controls can be used. Then the PCR products of the first round of PCR are subjected to a second round of PCR, and the forward primer used in the second round can be the same as in the first round; however, the reverse primer used in the second round is a different nested reverse primer than the reverse primer used the first round. Additionally, the first and second round primers can be exon-exon junction primers. The PCR products of the second round can be quantified to determine the amount of growth hormone mRNA in a sample.

In one aspect, the sample used is a blood sample, but other samples could be used as well. In one aspect, the sample is a human sample.

In one aspect, the reverse primer for nested PCR of the second round has the sequence of SEQ ID NO:4 (Nest_GH_rev). In one aspect, the forward primer has the sequence of SEQ ID NO:2 (BK158). In one aspect, for controls, “No-Template Controls” are used for comparison to the sample. In one aspect, when proteinase K digestion is being performed during purification the sample is vortexed for about 15 seconds.

In one aspect, the first round of PCR comprises at least about 25 cycles of PCR and the second round of PCR comprises at least about 25 cycles of PCR.

In one aspect, the primers are complementary to mRNA of growth hormone isoforms 1 and 2.

In one aspect, the PCR products are characterized by at least one of: melting curve analysis; agarose gel electrophoresis; and sequencing.

In one aspect, the method comprises the use of nested PCR and growth hormone mRNA amplification.

In one aspect, amplifications produce amplicons of 170 bp, with melting temperatures of 85.5° C., with the predicted sequence of growth mRNA (for example, see GenBank Accession No. NM_{—}000515.3; SEQ ID NO:7) or a fragment thereof.

In one embodiment, the present invention provides compositions and methods wherein a reverse-transcription quantitative nested PCR assay using the methods of the invention is linear from about 90 to about 90,000 copies/reaction of a synthetic oligonucleotide standard. In another embodiment, RNA is amplified enzymatically, but not using reverse transcriptase.

In one aspect, the amount of growth hormone mRNA in a sample from a subject with acromegaly prior to surgery or treatment ranges from about 1,000 copies/mL blood to about 30,000 copies/mL blood. In one aspect, the amount of growth hormone mRNA in a sample from a subject with acromegaly after surgery but still receiving treatment ranges from about 2,200 copies/mL blood to about 50,000 copies/mL blood. In one aspect, the amount of growth hormone mRNA in a subject receiving growth hormone replacement therapy for hypopituitarism is from about 350 copies/ml blood to about 9,500 copies/ml blood. In one aspect, the growth hormone mRNA is derived from B cells in blood. In one aspect, the method can quantify as few as about 90 copies of growth hormone mRNA per mL of blood.

The present invention encompasses the use of additional methods for detecting and measuring low levels of GH mRNA including, but no limited to, mapping and quantifying mammalian transcriptomes by RNA-Seq (Mortazavi et al., 2008, Nature Methods, 5:7:621) and by mass spectrometry of RNA (Meng and Limbach, 2006, Brief Funct. Genomic Proteomic, 5:1:87), the entire disclosures of which are incorporated by their entirety herein. For example, in one embodiment mRNA can be detected and quantified without first making a cDNA or amplifying the RNA without using reverse transcriptase.

The present invention further provides for administering exogenous growth hormone, or biologically active fragments, derivatives, analogs, or homologs thereof to a subject, or agonists or mimics thereof, detecting and measuring mRNA changes in the subject, detecting and monitoring changes in GH expression or levels or parts of the GH pathways, and monitoring the effects on the subject. The present invention further provides for comparisons of test results and levels with standard GH protein and GH mRNA levels of the invention in a sample obtained at a different time from the test subject, or from an otherwise identical test subject, or from a prepared standard.

The assay described herein is useful for determining whether growth hormone levels have increased or whether they have decreased in a subject, based on the amount of circulating miRNA regulated by growth hormone relative to a standard level or to a previously identified level in that subject, or to the amount of circulating growth hormone mRNA or protein. The assay further encompasses the use of one or more of the biomarkers used herein. The assay is useful for monitoring the progression of a disease or disorder with aberrant growth hormone levels as well as for monitoring treatment of such a subject. It can used to help modify the dosing regimen. In another aspect, the assay is useful for monitoring subjects to determine whether they have used or are using growth hormone, such as in a monitoring program for amateur or professional athletes where such use is prohibited.

When growth hormone is administered to a subject for treatment the amount administered is generally much less than the amount used by athletes. In one aspect, a treatment may range from about 0.1 to about 1.0 mg/day. In one aspect, athletes use doses of about 0.033 mg/kg/day to about 0.083 gm/kg/day, which in a 70 kg individual results in amounts of 2.3 mg/day and 5.8 mg/day, respectively.

In one embodiment, the present invention encompasses the use of detecting GH mRNA, measuring its changes, and comparing those changes in response to administration of an exogenous biologically active growth hormone, or a biologically active fragment, homolog, derivative, or analog thereof. In one aspect, the administered growth hormone is human growth hormone, or a biologically active fragment, homolog, derivative, or analog thereof. In one aspect, the administered growth hormone is synthetic, or a biologically active fragment, homolog, derivative, or analog thereof. In one aspect, the administered growth hormone is recombinant, or a biologically active fragment, homolog, derivative, or analog thereof.

Sequences of the invention include:

SEQ ID NO: 1
human growth hormone (217 amino acid residues)-
GenBank: AAA98618.1:
matgsrtslllafgllclpwlqegsafptiplsrlfdnamlrahrlhqla
fdtyqefeeayipkeqkysflqnpqtslcfsesiptpsnreetqqksnle
llrisllliqswlepvqflrsvfanslvygasdsnvydllkdleegiqtl
mgrledgsprtgqifkqtyskfdtnshnddallknygllycfrkdmdkve
tflrivqcrsvegscgf
SEQ ID NO: 2
ATCCAGGCTTTTTGACAACG (BK158: forward primer)
SEQ ID NO: 3
GGAGCAGCTCTAGGTTGGATT (BK369: reverse primer)
SEQ ID NO: 4
TGGAGGGTGTCGGAATAGAC (Nest_GH_rev)
SEQ ID NO: 5
ATGCTGGCGCTGAGTACGTC (GAPDH368: forward primer)
SEQ ID NO: 6
GGTGCAGGAGGCATTGCTGATG (GAPDH561: reverse primer).
SEQ ID NO: 7
human Growth Hormone mRNA, GenBank Accession No.
NM_000515.3 (822 bp):
aggatcccaaggcccaactccccgaaccactcagggtcctgtggacagct
cacctagctgcaatggctacaggctcccggacgtccctgctcctggcttt
tggcctgctctgcctgccctggcttcaagagggcagtgccttcccaacca
ttcccttatccaggctttttgacaacgctatgctccgcgcccatcgtctg
caccagctggcctttgacacctaccaggagtttgaagaagcctatatccc
aaaggaacagaagtattcattcctgcagaacccccagacctccctctgtt
tctcagagtctattccgacaccctccaacagggaggaaacacaacagaaa
tccaacctagagctgctccgcatctccctgctgctcatccagtcgtggct
ggagcccgtgcagttcctcaggagtgtcttcgccaacagcctggtgtacg
gcgcctctgacagcaacgtctatgacctcctaaaggacctagaggaaggc
atccaaacgctgatggggaggctggaagatggcagcccccggactgggca
gatcttcaagcagacctacagcaagttcgacacaaactcacacaacgatg
acgcactactcaagaactacgggctgctctactgcttcaggaaggacatg
gacaaggtcgagacattcctgcgcatcgtgcagtgccgctctgtggaggg
cagctgtggcttctagctgcccgggtggcatccctgtgacccctccccag
tgcctctcctggccctggaagttgccactccagtgcccaccagccttgtc
ctaataaaattaagttgcatca

The present invention further encompasses the sequences and their use.

Various aspects and embodiments of the invention are described in further detail below.

### BRIEF DESCRIPTION OF THE DRAWINGS

Example 1—

FIG. 1: Plasma miRNA Microarray Results

FIG. 2: Detection of human miRNAs present in unaltered plasma. miR-195 (blue lines), miR-223 (green lines), and miR-16 (red lines) were amplified from two plasma samples from the same volunteer, collected two weeks apart. The dashed lines are results from the first sample, the solid lines are from the second sample.

FIG. 3: Detection of nonhuman miRNA added to plasma. Solid lines (blue, green, and red) correspond to detection of cel-miR39 added to a plasma sample during purification (triplicate wells in array, thus three lines). Dashed lines (cyan, magenta, and yellow) correspond to plasma sample without addition of cel-miR39 during purification of RNA.

Example 2 Figures—

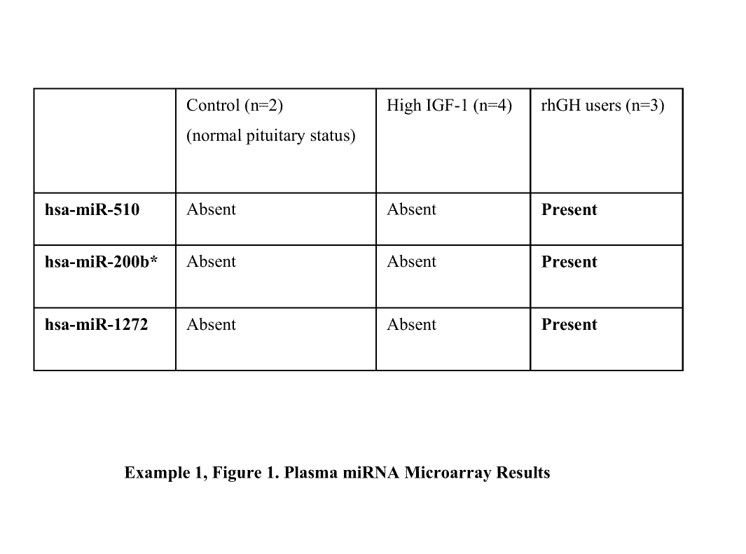

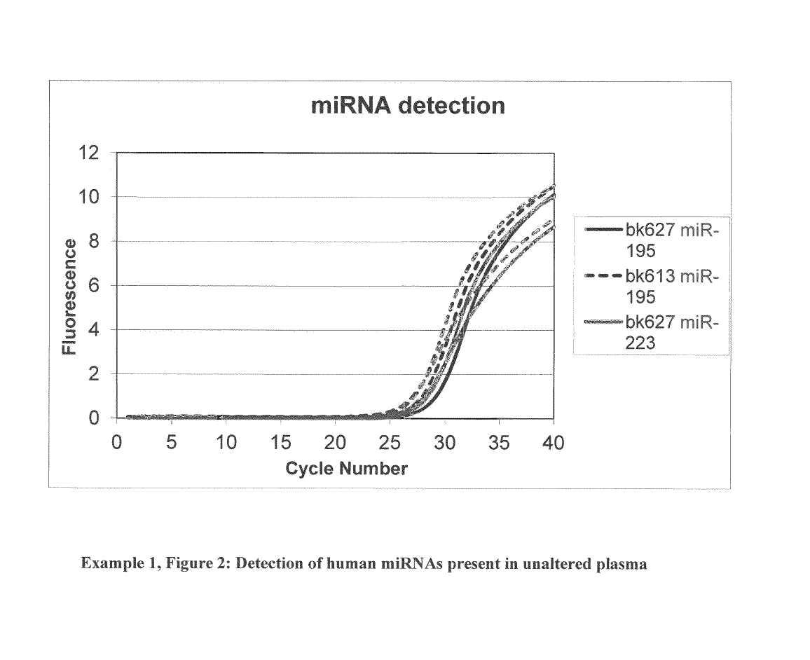

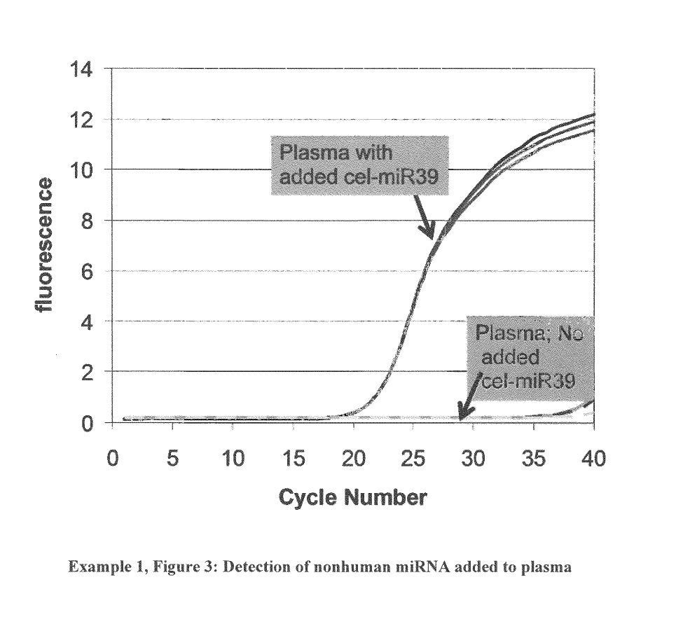

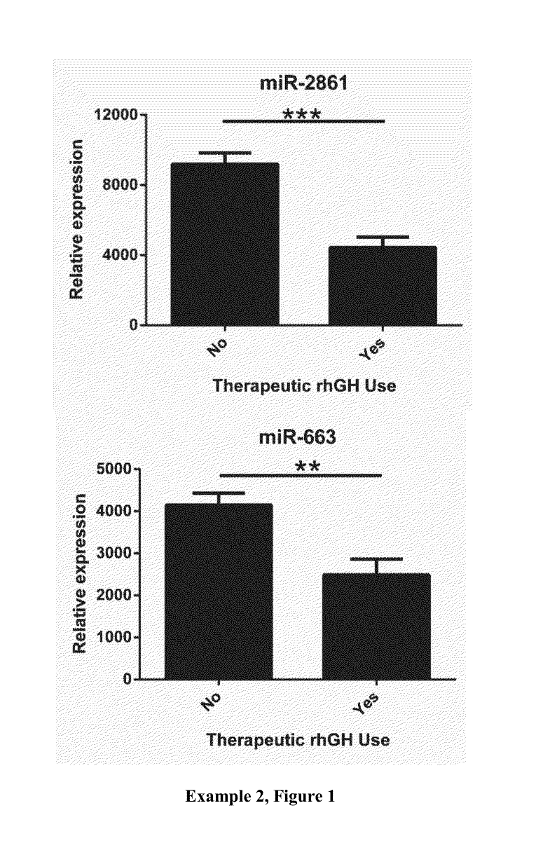

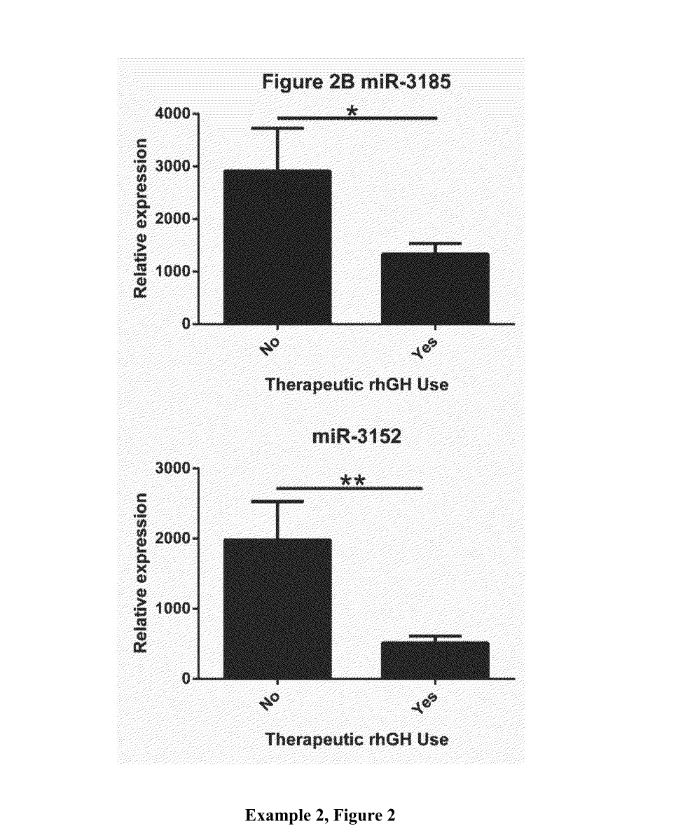

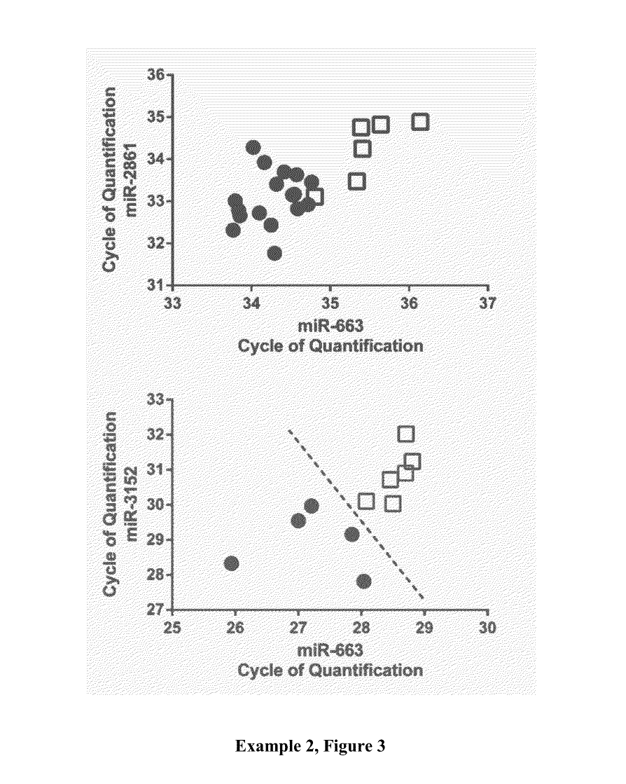

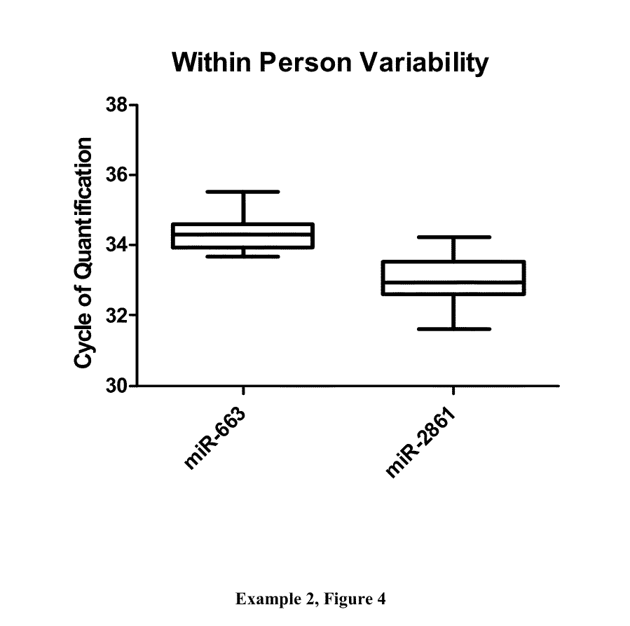

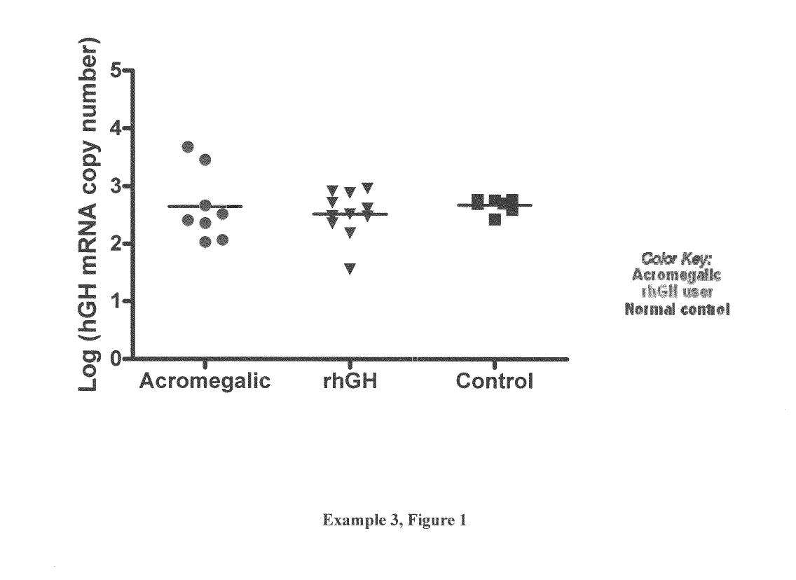

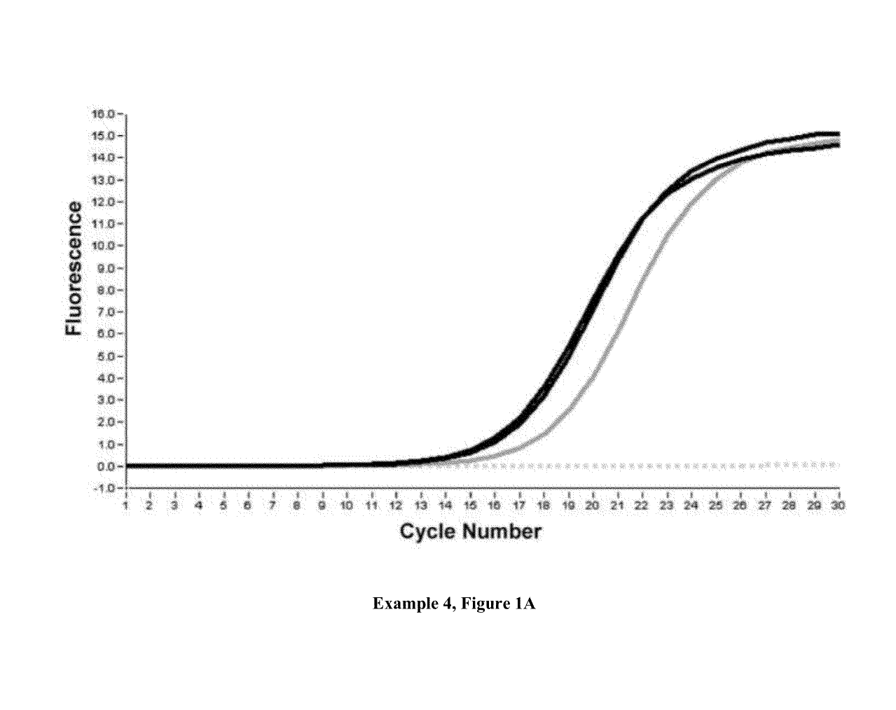

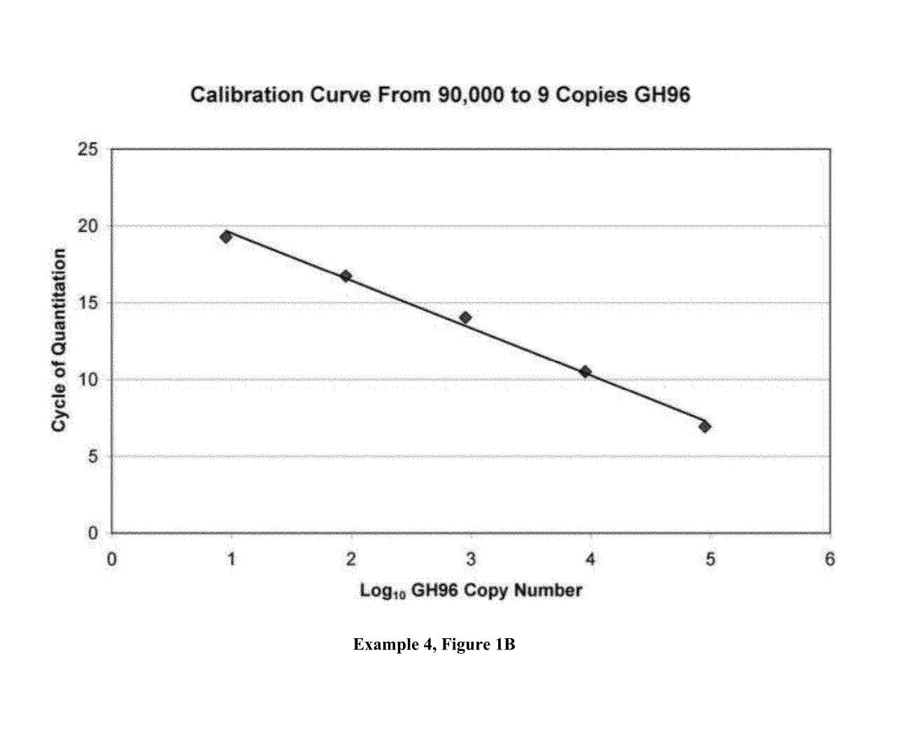

Example 2, FIG. 1: Panel A: Graph of relative expression of miR-2861 comparing therapeutic rhGH users to all others. Panel B: Graph of relative expression of miR-663 comparing therapeutic rhGH users to all others. Values are presented as mean±SEM, ***P<0.001**P<0.003.

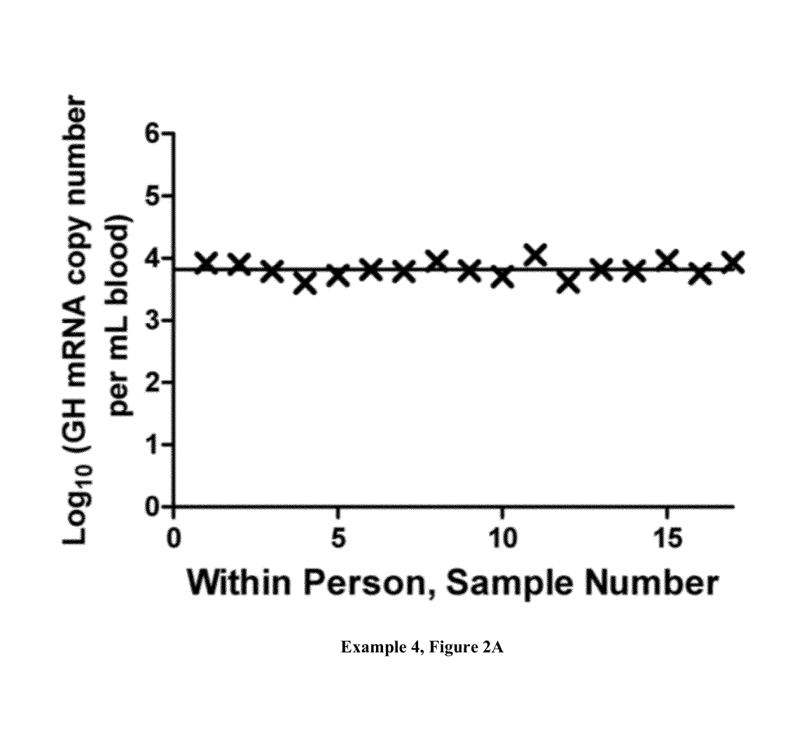

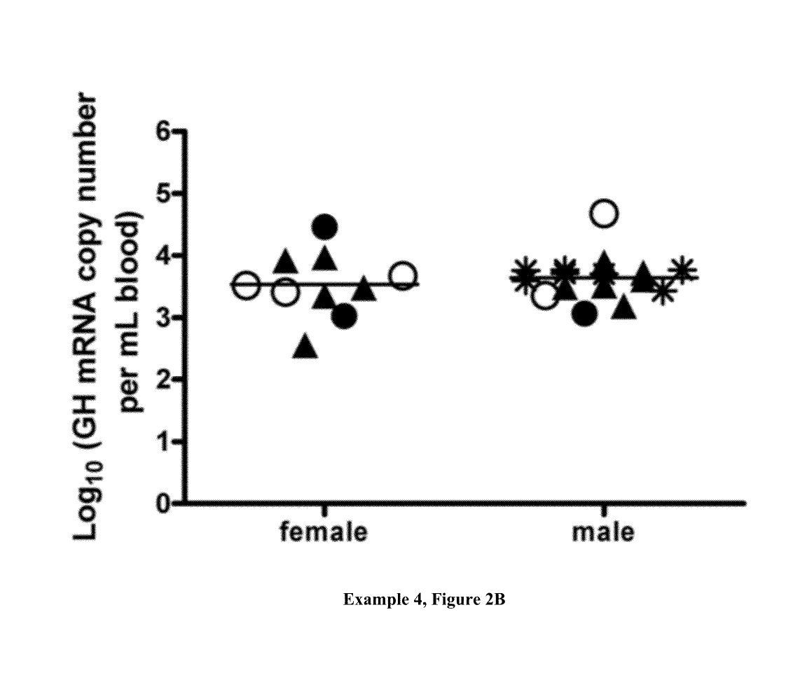

Example 2, FIG. 2: Panel A: Graph of relative expression of miR-3152 in preamplified samples comparing therapeutic rhGH users to controls. Panel B: Graph of relative expression of miR-3185 in preamplified samples comparing therapeutic rhGH users to controls. Values are presented as mean±SEM**P<0.003, *P<0.03.

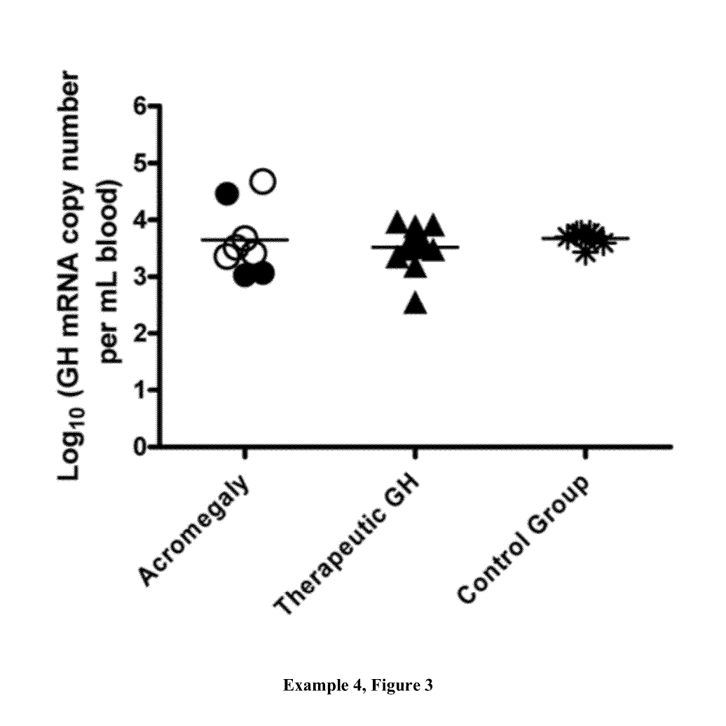

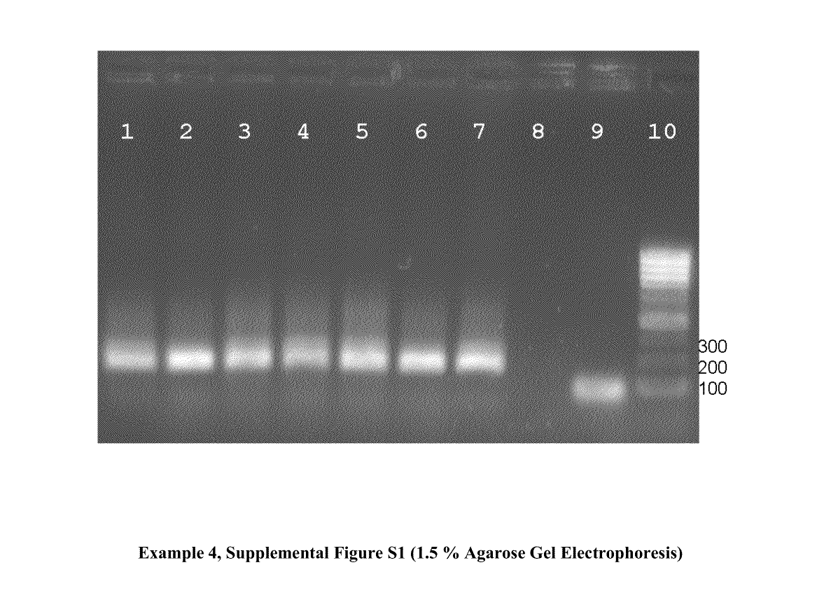

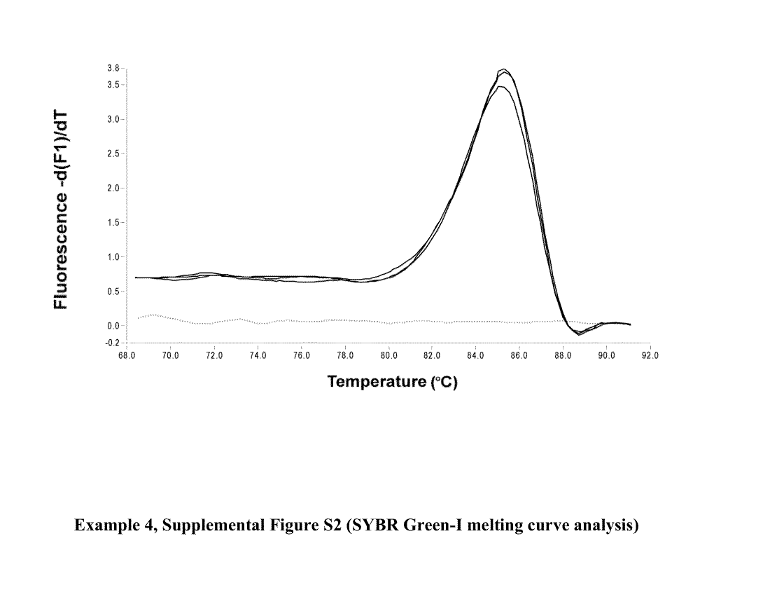

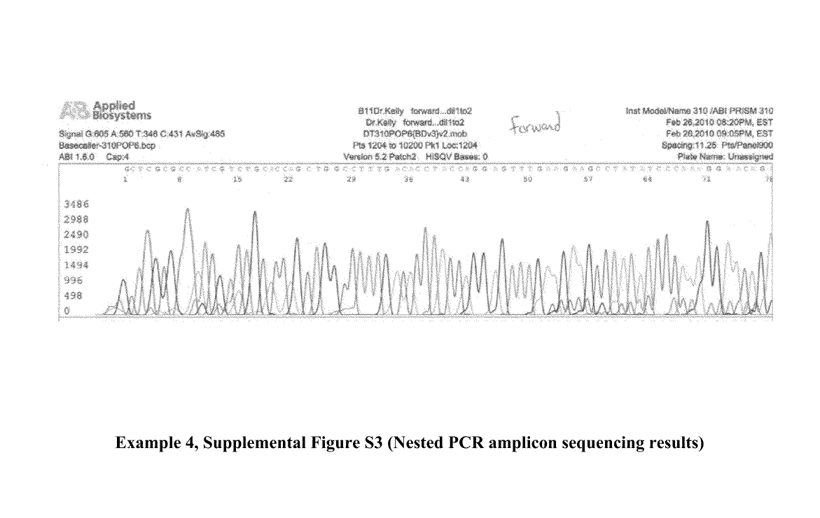

Example 2, FIG. 3: Potential differential markers of rhGH use. Panel A. miR-2861 versus miR-663: Results of qPCR on non-preamplifed cDNA illustrate a trend towards differential expression of circulating miRNA in rhGH users (open squares) and controls (closed circles). Panel B: miR-3152 versus miR-663: Results of qPCR on preamplified cDNA illustrate a difference in specific miRNAs between rhGH users (open squares) and controls (closed circles). The axes are in units of cycle of quantification (smaller numbers equates to higher concentration).

Example 2, FIG. 4: Intra-individual variability of miR-663 and miR-2861 in 15 samples collected over a period of 8 weeks. Data displayed as cycle of quantification for miR-663 and miR-2861. Smaller numbers equate to higher relative expression. The mean expression was 3780 (SD 1114) for miR-663 and 10100 (SD 4400) for miR-2861. (Box and whiskers min to max).

Example 3 Figures—

Example 3, FIG. 1. Biological Variation of hGH mRNA Serum Levels Within Pituitary Disorders and in Subjects Injected with rhGH. This figure graphically represents an experiment measuring circulating hGH mRNA in either acromegalics, subjects administered exogenous rhGH, or control subjects. The ordinate represents the log of hGH mRNA copy number.

Example 4 Figures—

Example 4, FIG. 1. A. Representative nested-primer real-time PCR amplifications of GH mRNA. Solid black lines, whole blood-derived RNA (two separate samples); solid grey line, pituitary-derived RNA; dashed line, negative control. B. Calibration curve with GH96 as calibrator. The assay is linear over at least a 10.000-fold range, from 9 copies to 90,000 copies.

Example 4, FIG. 2. GH mRNA copy numbers in whole blood. A. Samples from a single individual over the period of one year (symbol X). B. Samples from males and females. There was no statistically significant difference in copy numbers for males vs. females (P=0.55 for all subjects; 0.29 for patients with acromegaly; 0.80 for patients on rhGH). (Triangles, rhGH users; open circles, post treatment acromegaly; closed circles, pre-treatment acromegaly; asterisk, normal controls).

Example 4, FIG. 3. GH mRNA copy numbers in whole blood from patients and volunteers. Circles: Patients with acromegaly (pre-treatment closed circles, post-treatment open circles); triangles: patients receiving growth hormone replacement; asterisks: volunteers with no known pituitary disorders.

Example 4, Supplementary (“S”) FIG. 51 (1.5% Agarose Gel Electrophoresis). Amplicons from nested PCR targeting GH mRNA in whole blood specimens from a single volunteer collected over time are shown in lanes 1-7, no-template-control is shown in lane 8, amplicon from 90 copies of synthetic quantitation standard, GH96 is shown in lane 9, and 100 by ladder is in lane 10.

Example 4, Figure S2 (SYBR Green-I melting curve analysis). The melting curve analysis of products from a nested PCR targeting GH mRNA from whole blood (black lines) and pituitary (solid grey lines) and no-template control (dashed grey lines) demonstrate predicted melting temperatures of the products (85.5° C. for GH mRNA and no peak for no-template control).

Example 4, Figure S3 (Nested PCR amplicon sequencing results). The amplicons from nested PCR targeting GH mRNA were run on a 1.5% agarose gel, the bands were excised and submitted to a DNA sequencing facility. The sequencing results confirmed amplification of GH mRNA.

### DETAILED DESCRIPTION

### Abbreviations and Acronyms

CERA—continuous erythropoietin receptor activator
CQ—cycle of quantification
GAPDH—glyceraldehyde-3-phosphate dehydrogenase
GH—growth hormone
GHI™—growth hormone-inducible transmembrane protein
GHRH—growth hormone releasing hormone
hGH—human growth hormone
rGH—recombinant GH
rhGH—recombinant hGH
IGF-1—insulin-like growth factor 1
mRNA—messenger RNA
miRNA—microRNA
PCR—polymerase chain reaction
qPCR—quantitative PCR
RT—reverse transcriptase
USADA—United States Anti-Doping Agency
WADA—World Anti-Doping Agency

### DEFINITIONS

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

“Aberrant hGH” refers to levels, expression, or regulation of the hGH pathway that vary from normal, as in subjects with acromegaly or in subjects administered exogenous growth hormone or other regulators of growth hormone and its pathways/axis.

A “disease or disorder associated with aberrant hGH expression or levels” refers to a disease or disorder comprising either increased or decreased: hGH mRNA, hGH miRNA, or hGH protein expression or levels, and also includes aberrant regulation of the hGH pathways.

The term “about”, as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about”.

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As use herein, the terms “administration of” and or “administering” a compound should be understood to mean providing a compound of the invention or a prodrug of a compound of the invention to a subject in need of treatment.

The term “adult” as used herein, is meant to refer to any non-embryonic or non-juvenile subject. For example the term “adult adipose tissue stem cell,” refers to an adipose stem cell, other than that obtained from an embryo or juvenile subject.

Cells or tissue are “affected” by an injury, disease or disorder if the cells or tissue have an altered phenotype relative to the same cells or tissue in a subject not afflicted with the injury, disease, condition, or disorder.

As used herein, an “agonist” is a composition of matter that, when administered to a mammal such as a human, enhances or extends a biological activity of interest. Such effect may be direct or indirect.

A disease, condition, or disorder is “alleviated” if the severity of a symptom of the disease or disorder, the frequency with which such a symptom is experienced by a patient, or both, are reduced.

As used herein, “alleviating an injury, disease or disorder symptom,” means reducing the frequency or severity of the symptom.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

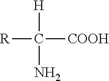

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

“Amplification” refers to any means by which a polynucleotide sequence is copied and thus expanded into a larger number of polynucleotide molecules, e.g., by reverse transcription, polymerase chain reaction, and ligase chain reaction.

As used herein, an “analog” of a chemical compound is a compound that, by way of example, resembles another in structure but is not necessarily an isomer (e.g., 5-fluorouracil is an analog of thymine).

An “antagonist” is a composition of matter that when administered to a mammal such as a human, inhibits or impedes a biological activity attributable to the level or presence of an endogenous compound in the mammal. Such effect may be direct or indirect.

The term “antimicrobial agents” as used herein refers to any naturally-occurring, synthetic, or semi-synthetic compound or composition or mixture thereof, which is safe for human or animal use as practiced in the methods of this invention, and is effective in killing or substantially inhibiting the growth of microbes. “Antimicrobial” as used herein, includes antibacterial, antifungal, and antiviral agents.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

A ligand or a receptor (e.g., an antibody) “specifically binds to” or “is specifically immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988, Antibodies, A Laboratory Manual, Cold Spring Harbor Publications, New York) for a description of immunoassay formats and conditions that can be used to determine specific immunoreactivity.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

The term “biological sample,” as used herein, refers to samples obtained from a living organism, including skin, hair, tissue, blood, plasma, cells, sweat, and urine.

As used herein, the term “biologically active fragments” or “bioactive fragment” of the polypeptides encompasses natural or synthetic portions of the full-length protein that are capable of specific binding to their natural ligand or of performing the function of the protein.

A “biomarker” is a specific biochemical in the body which has a particular molecular feature that makes it useful for measuring the progress of disease or the effects of treatment, or for measuring a process of interest.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize), as well as any of a number of characteristic structural and/or molecular features. A “cancerous” or “malignant cell” is understood as a cell having specific structural properties, lacking differentiation and being capable of invasion and metastasis. Examples of cancers are, breast, lung, brain, bone, liver, kidney, colon, and prostate cancer. (see DeVita, V. et al. (eds.), 2001, Cancer Principles and Practice of Oncology, 6th. Ed., Lippincott Williams & Wilkins, Philadelphia, Pa.; this reference is herein incorporated by reference in its entirety for all purposes).

“Cancer-associated” refers to the relationship of a nucleic acid and its expression, or lack thereof, or a protein and its level or activity, or lack thereof, to the onset of malignancy in a subject cell. For example, cancer can be associated with expression of a particular gene that is not expressed, or is expressed at a lower level, in a normal healthy cell. Conversely, a cancer-associated gene can be one that is not expressed in a malignant cell (or in a cell undergoing transformation), or is expressed at a lower level in the malignant cell than it is expressed in a normal healthy cell.

As used herein, the term “carrier molecule” refers to any molecule that is chemically conjugated to the antigen of interest that enables an immune response resulting in antibodies specific to the native antigen.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates or carboxylic acids, on the antigen and the carrier molecule. Chemical conjugation also includes non-covalent linkage between the antigen and the carrier molecule.

The term “competitive sequence” refers to a peptide or a modification, fragment, derivative, or homolog thereof that competes with another peptide for its cognate binding site.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.”

Similarly, it is known that a cytosine residue of a first nucleic acid strand is capable of base pairing with a residue of a second nucleic acid strand which is antiparallel to the first strand if the residue is guanine A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “complex”, as used herein in reference to proteins, refers to binding or interaction of two or more proteins. Complex formation or interaction can include such things as binding, changes in tertiary structure, and modification of one protein by another, such as phosphorylation.

A “compound,” as used herein, refers to any type of substance or agent that is commonly considered a drug, or a candidate for use as a drug, as well as combinations and mixtures of the above.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the term “diagnosis” refers to detecting cancer or a risk or propensity for development of cancer, for the types of cancer encompassed by the invention. In any method of diagnosis there exist false positives and false negatives. Any one method of diagnosis does not provide 100% accuracy.

A “disease” is a state of health of an animal wherein the subject cannot maintain homeostasis, and wherein if the disease is not ameliorated then the subject's health continues to deteriorate. In contrast, a “disorder” in an subject is a state of health in which the animal is able to maintain homeostasis, but in which the subject's state of health is less favorable than it would be in the absence of the disorder. Left untreated, a disorder does not necessarily cause a further decrease in the subject's state of health.

As used herein, an “effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly 5 amino acids or sugars in size. One skilled in the art understands that generally the overall three-dimensional structure, rather than the specific linear sequence of the molecule, is the main criterion of antigenic specificity.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

As used herein, a “functional” molecule is a molecule in a form in which it exhibits a property or activity by which it is characterized. A functional enzyme, for example, is one that exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCCS′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

The term “inhibit,” as used herein, refers to the ability of a compound, agent, or method to reduce or impede a described function, level, activity, rate, etc., based on the context in which the term “inhibit” is used. Preferably, inhibition is by at least 10%, more preferably by at least 25%, even more preferably by at least 50%, and most preferably, the function is inhibited by at least 75%. The term “inhibit” is used interchangeably with “reduce” and “block.”

As used herein “injecting or applying” includes administration of a compound of the invention by any number of routes and means including, but not limited to, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein the term “expression” when used in reference to a gene or protein, without further modification, is intended to encompass transcription of a gene and/or translation of the transcript into a protein.

“Malexpression” of a gene means expression of a gene in a cell of a patient afflicted with a disease or disorder, wherein the level of expression (including non-expression), the portion of the gene expressed, or the timing of the expression of the gene with regard to the cell cycle, differs from expression of the same gene in a cell of a patient not afflicted with the disease or disorder. It is understood that malexpression may cause or contribute to the disease or disorder, be a symptom of the disease or disorder, or both.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

The term “material” refers to any compound, molecule, substance, or group or combination thereof that forms any type of structure or group of structures during or after electroprocessing. Materials include natural materials, synthetic materials, or combinations thereof. Naturally occurring organic materials include any substances naturally found in the body of plants or other organisms, regardless of whether those materials have or can be produced or altered synthetically. Synthetic materials include any materials prepared through any method of artificial synthesis, processing, or manufacture. Preferably, the materials are biologically compatible materials.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present.

As used herein, the terms “native”, “natural” “native antigen”, or “natural antigen” refers to the antigen as it occurs in nature. With respect to the invention, the “native antigens” are of “low immunogenicity.” “Low immunogenicity” refers to the inability of the natural molecule to elicit a strong immune response resulting in the production of high affinity antibodies. The term “antigen”, “antigen of interest,” or specific molecules, such as the cancer-testis antigens encompassed herein include the whole molecule or any portions thereof that maintain antigenic distinctiveness specific for the native antigen.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

The term “Oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Operably linked” refers to a juxtaposition wherein the components are configured so as to perform their usual function. Thus, control sequences or promoters operably linked to a coding sequence are capable of effecting the expression of the coding sequence. By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

“Permeation enhancement” and “permeation enhancers” as used herein relate to the process and added materials which bring about an increase in the permeability of skin to a poorly skin permeating pharmacologically active agent, i.e., so as to increase the rate at which the drug permeates through the skin and enters the bloodstream. “Permeation enhancer” is used interchangeably with “penetration enhancer”.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “prevent,” as used herein, means to stop something from happening, or taking advance measures against something possible or probable from happening. In the context of medicine, “prevention” generally refers to action taken to decrease the chance of getting a disease or condition.

A “prophylactic” treatment is a treatment administered to a subject who does not exhibit signs of a disease or injury or exhibits only early signs of the disease or injury for the purpose of decreasing the risk of developing pathology associated with the disease or injury.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates. The terms “protein pathway” and “protein regulatory pathway” are used interchangeably herein.

The term “regulate” refers to either stimulating or inhibiting a function or activity of interest.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

As used herein, the term “secondary antibody” refers to an antibody that binds to the constant region of another antibody (the primary antibody).

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

A “test sample”, as used herein, refers to a sample of blood or other biologic sample obtained from a test subject.

Used interchangeably herein are the following pairs of words (1) “detect” and “identify”; (2) “select” and “isolate”.

The term “standard,” as used herein, refers to something used for comparison. For example, a standard can be a known standard agent or compound which is administered or added to a control sample and used for comparing results when measuring said compound in a test sample. In one aspect, the standard compound is added or prepared at an amount or concentration that is equivalent to a normal value for that compound in a normal subject. Standard can also refer to an “internal standard,” such as an agent or compound which is added at known amounts to a sample and is useful in determining such things as purification or recovery rates when a sample is processed or subjected to purification or extraction procedures before a marker of interest is measured.

A “subject” of analysis, diagnosis, or treatment is an animal. Such animals include mammals, preferably a human.

As used herein, a “subject in need thereof” is a patient, animal, mammal, or human, who will benefit from the method of this invention.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

“Tissue” means (1) a group of similar cells united to perform a specific function; (2) a part of an organism consisting of an aggregate of cells having a similar structure and function; or (3) a grouping of cells that are similarly characterized by their structure and function, such as muscle or nerve tissue.

The term to “treat,” as used herein, means reducing the frequency with which symptoms are experienced by a patient or subject or administering an agent or compound to reduce the frequency with which symptoms are experienced.

As used herein, the term “treating” may include prophylaxis of the specific injury, disease, disorder, or condition, or alleviation of the symptoms associated with a specific injury, disease, disorder, or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

“Treating” is used interchangeably with “treatment” herein.

By the term “vaccine,” as used herein, is meant a composition which when inoculated into an animal has the effect of stimulating an immune response in the subject, which serves to fully or partially protect the subject against a disease or its symptoms. The term vaccine encompasses prophylactic as well as therapeutic vaccines. A combination vaccine is one which combines two or more vaccines, or two or more compounds or agents.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### Embodiments

Methods and compositions are disclosed herein which encompass the use of circulating GH-regulated microRNA as a biomarker for a disease or disorder or changes in endogenous hGH resulting from administration of an exogenous growth hormone, or a biologically active fragment, homolog, derivative, or analog thereof, or administration of any other substance regulating hGH or its pathways.

Various techniques and methods are useful in the practice of the invention and are not all described herein. Various samples, including blood, useful for the practice of the invention can be obtained from a test subject. A sample can be processed by known techniques in order to detect microRNA. Various analytical techniques include, but are not limited to, detecting, measuring, and comparing levels and changes in GH mRNA and in microRNA regulated by GH. Various statistical and computer analyses can be performed when processing data.

In one aspect, the present invention provides for the use of the biomarker GH and for GH-regulated microRNA, or homologs or fragments thereof. In one aspect, the presence of a biomarker identified by the methods of the invention, or a difference in the level of the biomarker relative to a normal control level for a relevant population or for the individual, is indicative of a disease, disorder, or condition or indicative of the previous administration of exogenous growth hormone of biologically active fragments or homologs thereof, or of regulators of growth hormone or its pathways. In one embodiment, the present invention provides diagnostic assays detecting, diagnosing, and monitoring diseases, disorders, and conditions associated with aberrant expression or regulation of hGH and its pathways, as well as administration of exogenous growth hormone, or biologically active fragments or homologs thereof, or administration regulators of growth hormone and its regulatory pathways using the biomarkers of the invention.

The major isoform of the human growth hormone is a protein of 191 amino acids and a molecular weight of 22,124 daltons. The structure includes four helices necessary for functional interaction with the GH receptor.

Genes for human growth hormone, known as growth hormone 1 (somatotropin) and growth hormone 2, are localized in the q22-24 region of chromosome 17 and are closely related to human chorionic somatomammotropin (also known as placental lactogen) genes. GH, human chorionic somatomammotropin, and prolactin belong to a group of homologous hormones with growth-promoting and lactogenic activity. The nucleic acid sequence for human growth hormone (GH1) can be found at NCBI Reference Sequence: NC_{—}000017.10. See also GenBank accession M13438 (Seeburg, DNA 1 (3), 239-249 (1982). The protein encoded by this gene is a member of the somatotropin/prolactin family of hormones which play an important role in growth control. The gene, along with four other related genes, is located at the growth hormone locus on chromosome 17 where they are interspersed in the same transcriptional orientation; an arrangement which is thought to have evolved by a series of gene duplications. The five genes share a remarkably high degree of sequence identity. Alternative splicing generates additional isoforms of each of the five growth hormones, leading to further diversity and potential for specialization. This particular family member is expressed in the pituitary but not in placental tissue as is the case for the other four genes in the growth hormone locus. Mutations in or deletions of the gene lead to growth hormone deficiency and short stature.

mRNA sequences for isoforms of growth hormone protein are available, for example, with the GenBank accession numbers NM_{—}022562.2, NM_{—}022561.2, NM_{—}022560.2, NM_{—}022559.2, and NM_{—}000515.3 (SEQ ID NO:7). Peptide sequences for growth hormone isoforms can also be found at the NCBI website using GenBank accession numbers NP_{—}072056.1, NP_{—}072055.1, NP_{—}072054.1, NP_{—}072053.1, and NP_{—}000506.2.

The present application discloses that circulating messenger RNA (mRNA) for growth hormone can be measured quantitatively in blood and that the concentration of that mRNA is highly variable in people taking recombinant human growth hormone or affected with disorders of growth hormone, such as production of growth hormone by a tumor. Methods and compositions are disclosed herein which encompass the use of circulating hGH mRNA as a biomarker. Various techniques and methods are useful in the practice of the invention. Various samples, including blood, useful for the practice of the invention can be obtained from a test subject. A sample can be processed by known techniques in order to detect hGH mRNA. Various analytical techniques include, but are not limited to, detecting, measuring, and comparing levels and changes in hGH mRNA. Various statistical and computer analyses can be performed when processing data.

In one aspect, the present invention provides for the use of the biomarker hGH, particularly hGH mRNA, or homologs or fragments thereof. In one aspect, the presence of a biomarker identified by the methods of the invention, or a difference in the level of the biomarker relative to a normal control level, is indicative of a disease, disorder, or condition or indicative of administration of exogenous growth hormone of biologically active fragments or homologs thereof, or of regulators of growth hormone or its pathways. In one embodiment, the present invention provides diagnostic assays detecting, diagnosing, and monitoring diseases, disorders, and conditions, as well as administration of exogenous growth hormone, or biologically active fragments or homologs thereof, or administration regulators of growth hormone and its regulatory pathways using the biomarkers of the invention.

The present invention further provides for the use of GH administration to aid in monitoring therapy or as a therapeutic agent. In one aspect, the GH is human. In one aspect, the GH has the sequence of SEQ ID NO:1, or a biologically active fragment, homolog, or derivative thereof.

### EXAMPLES

### Example 1

The following example provides the preliminary studies and results upon which Examples 2, 3, and 4 are partially based. This example describes a preliminary study to test the hypothesis that administration of an exogenous growth hormone, such as recombinant growth hormone, will cause changes in expression or levels of mRNA or proteins in the GH signaling pathway or of miRNA regulated in the pathway.

Methods and IRB Approved Pilot Project Study Design

After giving informed consent, each volunteer provided samples of both blood and saliva. Volunteers were selected from 2 populations, one comprised of individuals without known pituitary disorders and the second of individuals with known and/or suspected pituitary disorders, particularly those with disorders in growth hormone production. Samples were collected and processed according to manufacturers' directions (PaxGene for whole-blood, Streck Cell-Free RNA for plasma, and Oragene•RNA for saliva) obtaining whole blood-derived RNA or plasma-derived RNA and saliva-derived RNA.

RNA was used as a template for either (1) quantification of hGH mRNA in blood (75 or 100 μL, of blood per analysis) by use of a reverse-transcription quantitative nested PCR assay developed in our laboratory using LightCycler® technology or (2) analysis of miRNAs in plasma by use of (a) microarrays (purchased from Affymetrix®) to study the expression pattern in plasma of ˜1100 human miRNAs and (b) a qPCR method in our laboratory for quantification.

To evaluate within-person and between-person variability of hGH mRNA in blood respectively, two types of samples were studied: (a) 20 whole blood samples from a single volunteer obtained over the course of one year; and (b) 24 whole blood samples from 24 volunteers, each of whom provided a single sample (see also Examples 3 and 4).

TABLE 1
miRNA in circulation specific to volunteers taking rhGH.

| | Control (n = 2) | High IGF-1 | rhGH users |
|---|---|---|---|
| | (normal pituitary status) | (n = 4) | (n = 3) |
| hsa-miR-510 | Absent | Absent | Present |
| hsa-miR-200b* | Absent | Absent | Present |
| hsa-miR-1272 | Absent | Absent | Present |

*Fishers exact test P value = 0.012 when comparing 6 non-rhGH users to 3 rhGH users.

Methods

a. Collect blood into a container that is suitable for keeping miRNAs stable (e.g., Streck© Cell-Free RNA™ BCT).

b. Process blood (e.g., by centrifugation) to obtain plasma.

c. Purify miRNA from plasma (e.g., by use of commercially available reagents, such as Qiagen miRNeasy mini kit).

d. Measure miRNAs such as miR-510, miR-200b*, and miR-1272 by any available method, such as RT-qPCR or Affymetrix miRNA microarray.

e. In one aspect, the test for growth hormone use is positive when all three miRNAs are detectable by Affymetrix miRNA microarray or are increased above normal when measured quantitatively by quantitative assays such as RT-qPCR assays.

The approach may be modified in the future to obviate the need for some steps, such as the step of isolating RNA prior to measuring the miRNAs.

Results

PCR amplifications produced products with the size, melting temperature and sequences predicted for products of hGH mRNA. The reverse-transcription quantitative nested PCR assay was linear from <90 to >90,000 copies/reaction of a synthetic oligonucleotide standard (GH96). In 10 separate runs on 9 days, the mean slope of the regression line was −3.7 with an SD=0.19 (CV=5%, range: −3.26 to −3.85, R²>0.99) indicating stability of the calibration curve and PCR efficiency of 86% (SD 7%). The between-run imprecision (CV) results for two samples of blood RNA analyzed in each of 10 runs were 46% and 31% (at mean concentrations of 263 and 340 copies/100 μL of blood, respectively).

The within-person variation of hGH mRNA was less than threefold (range 395 to 1070 copies/100 μL blood, CV=30%) over the period of one year (n=20), with the highest concentration coming from a sample collected one day after a 500 milliliter blood-donation. The inter-individual variation of copy number amongst the control group (males ages 27-36 years old) also was less than three-fold (range 268 to 590 copies/100 μL blood; between-person variability CV of 24%). By contrast, among acromegalic individuals, the hGH mRNA varied more than 40-fold (range 108 to 4800 copies/100 μL blood). Among individuals taking rhGH, hGH mRNA varied 33-fold (range 36 to 836 copies/100 μL blood). The copy number of hGH mRNA was not significantly correlated with age of subjects (Spearman r=−0.2925, approximate P=0.147), but there was a suggestion that copy number showed greater variability among volunteers older than 61 years than within the group of younger individuals.

The plasma samples were used to measure miRNAs by use of microarrays and also by use of quantitative PCR (qPCR). For microarray studies of miRNA, we examined samples from 3 individuals taking rhGH, 4 individuals with high IGF-1 values (either pre-treatment or treatment failure acromegalics), and 2 normal controls. Each of the samples from patients treated with rhGH contained a set of three specific miRNAs, none of which was detectable in any of the other samples (P=0.012, Fisher's Exact Test, comparing 6 non-rhGH users to 3 rhGH users). This qualitative finding is being investigated by use of qPCR to provide quantitative measurements of these three miRNAs in these samples and in an additional larger set of samples. To validate our qPCR for quantification of miRNAs, we successfully amplified several miRNAs in plasma, including miR-16, miR-29b, miR-195, and miR-223.

See Table 1 and FIGS. 1, 2, and 3.

### Example 1

### Conclusions

The nested-primer RT-qPCR assay is able to quantify hGH mRNA in whole-blood with good precision (see also Examples 3 and 4). The concentrations of GH mRNA in blood show only limited variability within a stable individual over the course of a year, and the between-person variability among young individuals is similar to that seen within a person. These attributes of hGH mRNA are promising for application to doping and suggest that the RT-qPCR assay for hGH mRNA is ready to be tested in samples from healthy individuals exposed to rhGH. Finally, the preliminary studies identifying potential microRNA (miRNA) biomarkers of GH administration established the basis for Example 2. The data demonstrate the potential for novel expression changes that could be useful for determining whether athletes have been abusing GH and for monitoring therapy of patients who are being administered GH, or a fragment, homolog, or derivative thereof (see Examples 2-4).

### Example 1

### Bibliography

1. Mandel, P. and P. Metais, Les acides nucléiques du plasma sanguin chez l'homme. C R Seances Soc Biol Fil, 1948. 142(3-4): p. 241-3.
2. Tsang, J. C. and Y. M. Lo, Circulating nucleic acids in plasma/serum. Pathology, 2007. 39(2): p. 197-207.
3. Zhang, J., et al., Presence of donor- and recipient-derived DNA in cell-free urine samples of renal transplantation recipients: urinary DNA chimerism. Clin Chem, 1999. 45(10): p. 1741-6.
4. H. Thakkar, et al., Circulating Nucleic Acids in the Assessment of Endogenous Growth Hormone Production Annals of the New York Academy of Sciences, 2008. 1137 (Circulating Nucleic Acids in Plasma and Serum V): p. 58-65.
5. Schaefer, A., et al., Diagnostic and prognostic implications of microRNA profiling in prostate carcinoma. Int J Cancer, 2010. 126(5): p. 1166-76.
6. Ho KK, Nelson A E. Growth hormone in sports: detecting the doped or duped. Horm Res Paediatr. 2011; 76 Suppl 1:84-90. Epub 2011 Jul. 21.
7. Holt R I. Detecting growth hormone abuse in athletes. Anal Bioanal Chem 2011 August; 401(2):449-62. Epub 2011 May 18.

### Example 2

In this example, the experiments disclosed in Example 1 were extended.

Introduction:

Circulating microRNAs (miRNA) in plasma are being studied for use as a biomarker for a disease and as a marker of administration of pharmaceutical agents. Administration of human growth hormone (GH) is banned by sporting authorities, but it continues to be used by athletes attempting to gain an unfair advantage in athletic competition. Current methods for detection of GH use rely on immunoassay technology and are limited by a short time-frame in which detection is possible. We hypothesized that administration of GH would alter expression of circulating miRNAs and that any changes could be quantified.

To identify potential miRNA targets, we used miRNA microarrays on plasma samples obtained from (1) individuals with no known pituitary disorders, (2) patients with excess GH production, and (3) patients receiving therapeutic replacement doses of rhGH. Positive findings from microarray screening were confirmed by specific real-time reverse-transcriptase (RT) quantitative PCR (qPCR) assays on a total of 35 samples.

Ribonucleic acids are a large family of molecules that perform numerous functions in biology. Messenger RNA is probably the most well-known class of RNA, but there are numerous other classes of RNA, including ribosomal RNA and transfer RNA both of which are important in translation. A number of regulatory RNA species have also been identified, including small nuclear RNA, small interfering RNA, and, more recently, microRNA (miRNA).

miRNAs are short (19-25 nucleotides), single-stranded, non-protein-coding RNAs. miRNAs can have both positive and negative effects on translation of specific genes or sets of genes. While miRNAs are produced within a cell, they have been found in many body fluids, including plasma, urine, and saliva. The biological functions of these cell-free miRNAs are not fully characterized, but increasing evidence indicates that they act as signaling molecules and that their expression is regulated by factors involved in angiogenesis, inflammation, response to exercise, differentiation, growth, and other cellular functions.

Changes in individual miRNA concentrations have been identified in disease states and also as a result of administration of compounds, including a long-acting erythropoiesis stimulating peptide. Patients with growth hormone (GH)-secreting tumors have altered expression of miRNA in their pituitary. Because of interest in detecting doping with growth hormone we have asked whether administration of recombinant GH (rhGH) affects the expression of specific miRNAs in circulation.

### Methods

### Example 2

### Subjects and Samples

The study was designed to compare expression of miRNAs in subjects with and without rhGH administration. Within the group not administered rhGH, there were two groups: individuals without a known pituitary disorder, and individuals with known or suspected pituitary disorders (seen in the neuro-endocrinology clinic at the University of Virginia), specifically those with disorders in growth hormone production. With approval from the University of Virginia Institutional Review Board, volunteers provided informed consent, were enrolled, and provided a plasma specimen. Blood (10 mL) was collected into Streck Cell-Free RNA Blood Collection Tubes. Blood samples were collected by phlebotomists at the University of Virginia. We measured cell-free miRNA in plasma from 6 individuals receiving therapeutic replacement doses of rhGH (0.2-0.8 mg/day), 12 individuals with acromegaly (4 pre-treatment and 8 post-treatment), and 3 individuals with no evidence of abnormalities of the pituitary or of growth hormone secretion or action. For evaluation of within-person variability, one volunteer, with no known pituitary disease, provided 15 samples, collected at various times of day, over the period of two months.

RNA Extraction

The blood samples were processed rapidly after blood collection by separation of plasma from cellular components through centrifugation at 1600 rcf for 10 minutes at 25° C. The plasma was then aliquoted into 2-mL Eppendorf tubes and subjected to centrifugation at 16,000 rcf for 10 minutes at 25° C. The pellet was discarded and the supernatant was frozen at −20° C. Using 200 μL of plasma, miRNA was purified according to the Qiagen miRNeasy Serum/Plasma reagent set instructions. Purified miRNA was eluted with 14 μL of nuclease-free water, and samples were stored at −20° C.

miRNA Microarray

miRNA microarrays were performed utilizing Affymetrix® GeneChip® miRNA 2.0 Arrays following the manufacturer's protocol. The FlashTag™ Biotin HSR RNA Labeling Kit was used. Briefly, a Poly (A) tail is placed on the 3′ end of purified RNA. Subsequently, the Poly (A) tailed RNA undergoes a ligation reaction to append a biotin label to the target RNA sample. This biotinylated RNA molecule is hybridized to the miRNA array and resulting hybridization intensity is measured. Samples chosen for microarrays included 4 samples obtained from individuals with acromegaly (high IGF-1), 3 samples from therapeutic rhGH users (normal IGF-1), and 2 controls from individuals with no known pituitary disorders.

Reverse Transcription of miRNA and Quantitative PCR

Reverse transcription was performed using the Qiagen miScript II RT reagent set. Four microliters of miRNA were added to the HiSpec Buffer, nucleic acid mix, and reverse transcriptase, with a final volume of 20 μL. The reverse transcription reaction went for 60 minutes at 37° C., followed by a denaturation step for 5 minutes at 95° C. The resulting cDNA was stored at −20° C. until use. Four microliters of the cDNA was diluted ten-fold with 400 μL of nuclease-free water to be used as template in quantitative PCR (qPCR). Qiagen miScript SYBR Green PCR Kit was used for the qPCR following the manufacturer's protocol on an Applied Biosystems StepOnePlus™ PCR system. Cycling conditions were: 15-minute incubation at 95° C. followed by 40 cycles of: denaturation for 15 seconds at 94° C., annealing for 30 seconds at 55° C., and extension for 30 seconds at 70° C. with fluorescent signal acquisition at the end of extension. Each sample was subjected to analysis of 7 different miRNA targets as well as RNA extraction, reverse transcription, and no-template, controls.

Quantification was based upon the addition of a synthetic standard miRNA. Prior to RNA purification 5.6*10̂8 copies of the nonhuman miRNA cel-miR-39 were added to each plasma sample. After purification and at the start of the qPCR reaction, there are 1.5*10̂6 copies of cel-miR-39 present. Thus, cel-miR-39 acts as an extraction and normalization control and allows for estimation of the targeted miRNA expression in a sample by calculating a 10-fold copy number difference per 3.3 PCR cycles.

Preamplification of cDNA

Because miRNA concentrations in plasma are low compared to miRNA obtained from cellular sources, a preamplification step was performed on 5 samples from controls and 6 samples from therapeutic rhGH users. The Qiagen miScript PreAMP reagent set was used to increase the sensitivity of the quantitative PCR assay. The manufacturer's directions were followed, but since there no known publications that have used this reagent set, the method will be described here in more detail. Briefly, 2 μL of cDNA was diluted with 8 μL nuclease-free water. 5 μL of the diluted cDNA was added to 2 μL of HotStarTaq DNA Polymerase, 5 μL of miScript PreAMP Custom Primer Mix, 7 μL of RNase-free water, 1 μL miScript PreAMP Universal Primer, and 5 μL 5× miScript PreAMP Buffer. The resulting solution was subject to the following cycling conditions: 15 minutes at 95° C. for the initial activation step, followed by a 2-step cycling procedure repeated for 12 cycles: denaturation occurred for 30 seconds at 94° C. and annealing/extension for 3 minutes at 60° C. When cycling was complete, the product was stored at −20° C. until qPCR. Immediately prior to qPCR, the products of the preamplification process were diluted 20 fold by the addition of 475 μL of nuclease-free water, and 2 μL of the diluted, preamplified cDNA was used as template for qPCR as described above.

PCR Product Characterization

In addition to quantitative analysis of PCR product amplification using SYBR Green I fluorescence, RT-PCR products were characterized by melting curve analysis which confirmed that all products had the predicted melting temperatures.

Statistical Methods

Statistics where calculated either by LPE, LIMMA, or by non-parametric analysis in GraphPad Prism version 5.

### Results

### Example 2

### Microarray Results

The microarray used for this part of the study is designed to measure, if present, 1105 human miRNAs. In the plasma samples analyzed by this technique, 317 of the 1105 miRNAs were found in at least one sample and 41 miRNAs were commonly expressed in all 9 samples subjected to microarray analysis. When analyzing the quantitative microarray results with LPE and LIMMA, four miRNAs, miR-663, miR-2861, miR-3152, and miR-3185, showed statistically significant changes in expression in the volunteers who used rhGH as part of their therapy regimen.

RT-qPCR Results

For the RT-qPCR studies, we focused on the 4 miRNAs identified in the microarray study as candidate markers of rhGH administration. We also measured 3 additional miRNAs that appeared to be possibly altered by rhGH but were not identified by LPE and LIMMA as statistically significantly differently expressed (miR-510, 200b*, and miR-1272). We performed RT-qPCR assays of these 7 miRNAs in the larger group of 35 samples from volunteers who had or had not received rhGH.

The RT-qPCR assays were performed initially without preamplification. The concentrations of 2 of the 7 miRNAs were statistically significantly lower in samples from patients who had received rhGH compared with those who had not received the hormone. In the rhGH user group the mean concentration of miR-2861 was 9.46*10̂7 copies per milliliter of plasma (SD 4.36*10̂7 copies) versus 1.76*10̂8 copies per milliliter of plasma (SD 1.04*10̂8 copies) in all others (Mann-Whitney P value=0.0004) (Example 2, FIG. 1A). The mean concentration of miR-663 in the rhGH users was 5.29*10̂7 copies per milliliter of plasma (SD 2.76*10̂7 copies) and 8.41*10*7 copies per milliliter of plasma (SD 4.49*10̂7 copies) in control (Mann-Whitney P value=0.002) (Example 2, FIG. 1B).

Because of the low concentrations of miRNAs in plasma compared to cells, we performed additional studies using pre-amplification of targeted miRNAs to improve measurement of all miRNAs (see Methods). For these studies, we reanalyzed 5 control samples and 6 therapeutic rhGH user samples subject to preamplification and subsequent RT-qPCR. Four of these samples had been used in the initial microarray studies, (one control and three therapeutic rhGH users) and the rest had not been. Results after a pre-amplification step confirmed the changes of miR-2862 and miR-633 seen in the rhGH group in the microarray and in the initial RT-qPCR studies. Moreover, the testing demonstrated decreased concentrations of 2 additional miRNAs, miR-3152 and miR-3185, in rhGH group (Example 2, FIG. 2). Thus all four miRNAs identified as having statistically significant different expression in the microarray study were confirmed by RT-qPCR. By contrast, none of the 3 other miRNAs that appeared in the microarray data to be marginally affected by rhGH were found to be affected when the samples were analyzed by RT-qPCR (data not shown). In summary, the mean concentrations of miR-2861, miR-663, miR-3152, and miR-3185 were lower in therapeutic rhGH users than in the others (P=0.004, 0.004, 0.004, and 0.022, respectively) (Example 2, FIGS. 1 and 2).

Detection of drug use often requires analysis of more than one marker. Thus we performed exploratory bivariate analyses of the expression of miR-663, miR-2861 and miR-3152. Bivariate analysis of miR-663 and miR-2861 showed a promising separation of therapeutic rhGH users and controls in the set of samples analyzed without preamplification (Example 2, FIG. 3A). The same approach using miR-663 and miR-3152 allowed a clear separation of therapeutic rhGH users and controls in the group of subjects tested by RT-qPCR after preamplification (Example 2, FIG. 3B).

Because changes of marker concentrations over time can prove to be important for detection of abuse of drugs by athletes, we investigated the within-person variability of miR-663, miR-2861, and miR-3152 in 15 samples collected from a single individual over the period of 2 months. The results show that the mean expression is 3780 (SD 1114) for miR-663 and 10100 (SD 4400) for miR-2861 (Example 2, FIG. 4). These two miRNAs, miR-663 and miR-2861, look promising for inclusion into longitudinal monitoring of athletes being tested for rhGH use.

### Discussion

### Example 2

This is the first study to identify candidate miRNA markers of rhGH use. By utilizing two separate methods, namely microarrays and RT-qPCR, and clinically well-characterized patients, we have identified miRNAs that have statistically significant differences in expression in controls versus individuals receiving therapeutic replacement doses of rhGH (0.2-0.8 mg/day). An earlier study by Leuenberger and colleagues showed the utility of circulating miRNA to detect use of another performance enhancing agent, the long-acting erythropoiesis stimulating agent mirCERA. This current study and that of Leuenberger suggest that measurement of miRNAs may find a role in detection of banned performance-enhancing substances.

In studies such as this, it is reasonable to ask whether the identified miRNAs are plausibly related to GH administration. Several of the miRNAs have interesting relations to GH. miR-1272 is predicted to target growth hormone-inducible transmembrane protein (GHI™). miR-2861 is predicted to target PTTG11P pituitary tumor-transforming 1 interacting protein. miR-3152 reportedly targets IGF-1 Binding Protein 7, a potential downstream marker of GH use. miR-3185 reportedly targets GDF 11 growth differentiation factor 1114. By contrast to these miRNAs, neither miR-510 nor miR-663 has known functions directly related to the physiology of GH. In view of the relative youth of the miRNA field, it is reasonable to expect that further relationships between GH and miRNAs will be elucidated, and the rhGH associated discriminative function of miR-663 reported here suggest that it is worth study in the context of rhGH action.

The set of samples tested by RT-qPCR was more than 4 times as large as the set used for the microarray studies, thus confirming the results of the screening test. Moreover, comparison of results of this study and subsequent studies is aided by the use of a quantitative RT-PCR assay and by making all of the raw data available for data-level meta-analyses.

The study includes several strengths. The samples were all collected in the same time period using the same collection technique and the same lot number of blood-collection tubes. This avoids artifacts that occur from failure to control collection. The volunteers enrolled in the neuro-endocrinology clinic are all well-characterized patients. Samples from controls and patients were purified at the same time using the same reagent set, and all samples were analyzed together. Moreover, the conclusions do not rest on the results of the screening (microarray) test, but are based on quantitative (RT-qPCR) assays. As shown here, not all differences that appear to exist on microarray can be confirmed by a quantitative method. Finally, the apparent lack of significant effect of rhGH on several miRNAs may be useful in interpreting future studies, especially microarray screening studies, which are susceptible to false identification of candidate markers.

Unauthorized use of human growth hormone is banned by international sporting authorities. Regardless, many rogue athletes presumably use rhGH for its lipolytic, ergogenic, and perceived recovery effects. A high-sensitivity immunoassay has been developed that allows detection of rhGH administration for a period of up to roughly 24 hours. A recently implemented approach that combines protein biomarkers of rhGH use will increase the window of detection over the isoform-based immunoassay method. Regardless, there is still a need for improved detection of GH use in sports and this research describes early efforts to develop a new assay using circulating miRNA as a marker of rhGH use.

The natural progression of this research is to administer larger doses of rhGH to athletic volunteers in a manner that more closely replicates methods used by rogue athletes. This will require collection of pre-dose samples, followed by administration of one of several doses of rhGH with further sample collections, and finally, a wash-out phase to determine how long rhGH administration is detectable.

### Example 2

### Bibliography

1. Bartel D P. MicroRNAs: genomics, biogenesis, mechanism, and function. Cell. Jan. 23, 2004; 116(2):281-297.
2. He L, Hannon G J. MicroRNAs: small RNAs with a big role in gene regulation. Nat Rev Genet. July 2004; 5(7):522-531.
3. Weber J A, Baxter D H, Zhang S, et al. The MicroRNA Spectrum in 12 Body Fluids. Clin Chem. Nov. 1, 2010 2010; 56(11):1733-1741.
4. Baggish A L, Hale A, Weiner R B, et al. Dynamic regulation of circulating microRNA during acute exhaustive exercise and sustained aerobic exercise training J. Physiol. Aug. 15, 2011; 589(Pt 16):3983-3994.
5. Pfaff N, Moritz T, Thum T, et al. miRNAs involved in the generation, maintenance, and differentiation of pluripotent cells. J Mol Med (Berl). Jun. 9, 2012.
6. Cheng A M, Byrom M W, Shelton J, et al. Antisense inhibition of human miRNAs and indications for an involvement of miRNA in cell growth and apoptosis. Nucleic Acids Res. 2005; 33(4):1290-1297.
7. Zhang Y, Liu D, Chen X, et al. Secreted monocytic miR-150 enhances targeted endothelial cell migration. Mol Cell. Jul. 9, 2010; 39(1):133-144.
8. Cortez M A, Bueso-Ramos C, Ferdin J, et al. MicroRNAs in body fluids—the mix of hormones and biomarkers. Nat Rev Clin Oncol. August 2011; 8(8):467-477.
9. Leuenberger N, Jan N, Pradervand S, et al. Circulating microRNAs as long-term biomarkers for the detection of erythropoiesis-stimulating agent abuse. Drug Test Anal. Nov. 24, 2011.
10. Mao Z G, He D S, Zhou J, et al. Differential expression of microRNAs in GH-secreting pituitary adenomas. Diagn Pathol. 2010; 5:79.
11. Jain N, Thatte J, Braciale T, et al. Local-pooled-error test for identifying differentially expressed genes with a small number of replicated microarrays. Bioinformatics. Oct. 12, 2003; 19(15):1945-1951.
12. Smyth G K. Linear models and empirical bayes methods for assessing differential expression in microarray experiments. Stat Appl Genet Mol Biol. 2004; 3:Article3.
13. Sottas P E, Robinson N, Rabin O, et al. The athlete biological passport. Clin Chem. July 2011; 57(7):969-976.
14. Wang X. miRDB: a microRNA target prediction and functional annotation database with a wiki interface. RNA. June 2008; 14(6):1012-1017.
15. Bidlingmaier M, Suhr J, Ernst A, et al. High-Sensitivity Chemiluminescence Immunoassays for Detection of Growth Hormone Doping in Sports. Clin Chem. Mar. 1, 2009 2009; 55(3):445-453.
16. Erotokritou-Mulligan I, Eryl Bassett E, Cowan D A, et al. The use of growth hormone (GH)-dependent markers in the detection of GH abuse in sport: Physiological intra-individual variation of IGF-I, type 3 pro-collagen (P-III-P) and the GH-2000 detection score. Clin Endocrinol (Oxf). April 2010; 72(4):520-526.

### Example 3

Detection of human growth hormone use is very challenging due to the short half-life of circulating human growth hormone and the fact that the recombinant human growth hormone used is identical in protein sequence as the native, naturally produced growth hormone. The present example discloses that circulating messenger RNA (mRNA) for growth hormone can be measured quantitatively in blood and that the concentration of that mRNA is highly variable in people taking recombinant human growth hormone or affected with disorders of growth hormone, such as production of growth hormone by a tumor.

Methods

a. Collect blood into a container that is suitable for keeping whole blood mRNA stable (e.g., PaxGene™ Blood RNA tube).

b. Process blood (e.g., by centrifugation) to obtain lymphocytes.

c. Purify mRNA from lymphocytes (e.g., by use of commercially available reagents, such as PaxGene Blood RNA kit).

d. Measure hGH mRNA by any available quantitative method, such as RT-qPCR with nested primer technology

e. The test for growth hormone use is positive when hGH mRNA is above or below normal when measured quantitatively by quantitative assays such as RT-qPCR assays.

Variation of hGH mRNA Among Patients with Pituitary Disorders

In one study, the samples used included 19 whole blood samples from 19 volunteers with pituitary disorders, 7 whole blood samples from 7 volunteers without pituitary disorders, each of whom provided a single sample.

In acromegalic individuals, the hGH mRNA varied more than 40-fold (range 108 to 4800 copies/100 μL, blood) (see FIG. 1). In individuals taking rhGH, hGH mRNA varied 33-fold (range 36 to 836 copies/100 μL blood). In the control group (males ages 27-36 years old) hGH mRNA less than three-fold (range 268 to 590 copies/100 μL blood).

### Example 3

### Bibliography

1. Thakkar H, Butt A N, Powrie J, Holt R, Swaminathan R., “Circulating nucleic acids in the assessment of endogenous growth hormone production”, Ann N Y Acad Sci. 2008 August; 1137:58-65.
2. Ho KK, Nelson A E., “Growth hormone in sports: detecting the doped or duped”, Horm Res Paediatr. 2011; 76 Suppl 1:84-90. Epub 2011 Jul. 21.
3. Holt R I, “Detecting growth hormone abuse in athletes”, Anal Bioanal Chem 2011 August; 401(2):449-62. Epub 2011 May 18.
4. hGH isoform differential immunoassays for anti-doping analyses. World Antidoping Agency (see their website).

### Example 4

The following example discloses an assay that is a great improvement over previous assay, particularly in sensitivity for identifying and quantifying low levels of GH mRNA. We describe the development and characterization of an assay to quantify low concentrations of growth hormone mRNA in blood and pituitary tissue and the use of the assay to measure GH mRNA in blood from patients with increased or decreased pituitary production of GH and from individuals with no evidence of a pituitary disorder.

### Methods

### Example 4

### Subjects and Samples

The study was designed for comparison of two groups: individuals without a known pituitary disorder, and individuals with known or suspected pituitary disorders (seen in the neuro-endocrinology clinic at the University of Virginia), particularly those with disorders in growth hormone production. With approval from the University of Virginia Institutional Review Board, volunteers were enrolled and provided both whole-blood and saliva specimens. Blood specimens (2.5 mL) were collected in PAXgene Blood RNA Tubes, and saliva was collected by use of Oragene•RNA for Expression Analysis Self-Collection Kits. Blood samples were collected by phlebotomists at the University of Virginia and saliva samples were self-collected by the volunteers immediately prior to blood collection.

To evaluate between-person variability of GH mRNA in blood we enrolled: 8 individuals with acromegaly, 10 individuals receiving therapeutic replacement doses of recombinant GH (rhGH) (0.2-0.8 mg/day) as therapy (treated/administered rGH), one of whom provided 2 samples, and 7 individuals with no evidence of a pituitary disorder (controls). For evaluation of within-person variability, one additional volunteer, with no known pituitary disease, provided 17 samples, collected at various times of day, over the period of one year.

### RNA Extraction and Characterization

The whole blood samples were stored at room temperature for a minimum of two hours (per manufacturer's directions). Immediately after the room temperature incubation, RNA was purified according to PAXgene Blood RNA Kit instructions. The sole modification was the use of a 15-second vortexing for mixing during the proteinase K digestion [9]. Purified RNA was eluted with 100 μL elution buffer and samples were stored at −20° C. or −80° C. until spectrophotometric analysis for nucleic acid quantification and assay by RT-qPCR. The purified samples had an average RNA concentration of 100 ng/μL and an average A260/A280 ratio of 2.17 (Nanodrop 1000). For studies of plasma, blood samples were collected in Streck Cell-Free RNA tubes and processed rapidly after blood collection by centrifugation at 1600 rcf for 10 minutes at 25° C. The plasma was then aliquoted into 2-mL Eppendorf tubes and subjected to centrifugation at 16,000 rcf for 10 minutes at 25° C. The pellet was discarded and the supernatant was frozen at −20° C. Essential MIQE guidelines were followed during sample preparation and assay development.

RT-qPCR of GH and GAPDH

Reverse transcription and quantitative PCR were performed using the Roche LightCycler® Carousel-Based System. For reverse transcription of GH mRNA (HUGO GH1 and accession NM_{—}000515.3; SEQ ID NO:7) and the first 25 PCR cycles, as well as reverse transcription and amplification of glyceraldehyde-3-phosphate dehydrogenase (GAPDH) mRNA, we used the Roche LightCycler® RNA Master SYBR Green I reagent set. For GH mRNA, 3 μL of purified total RNA was used as template for the reverse transcription reaction and initial PCR cycles, using primers described below; 1 μL of purified total RNA was used as template for GAPDH mRNA reverse transcription and subsequent amplification. Reverse transcription of GH and GAPDH utilized Tth polymerase, which was present in a 1× concentration (overall reaction volume of 10 μL), and reverse transcription occurred for 20 minutes at 61° C. Following reverse transcription, for GH mRNA, 25 cycles of PCR were performed with these cycling conditions: 1 second at 92° C., 1 second at 59° C., and 5 seconds at 72° C. Fluorescent signal from SYBR Green I was acquired at 80° C. after a 2-second delay. Following reverse transcription, for GAPDH mRNA, 35 cycles of PCR were performed with these cycling conditions: 1 second at 95° C., 1 second at 59° C., 5 seconds at 72° C., and fluorescent signal was acquired at 80° C. after a 2 second delay.

Nested PCR for GH mRNA Amplification and Quantification

To increase analytical specificity and sensitivity, we used 3 μL of product from the first 25 PCR cycles as template for a second round of PCR, using the same forward primer (SEQ ID NO:2; BK158; 300 nmol/L) and a new reverse primer (SEQ ID NO:4; Nest_GH_Rev; 300 nmol/L) that had a binding site within the amplicon produced from the original primers. Thirty more cycles of PCR, using LightCycler® FastStart DNA Master SYBR Green I reagent (Roche), were then performed with a single enzyme activation cycle of 15 minutes at 95° C., followed by 30 cycles of 1 second at 92° C., 1 second at 59° C., and 5 seconds at 72° C. Fluorescence signal from SYBR Green I was acquired at 80° C. after a 2-second delay.

Throughout assay development and data collection, no-template-control (NTC) samples were included in each experiment to test for contamination. NTCs had no detectable signals.

Primers were designed to be exon-exon junction spanning to increase specificity for mRNA rather than genomic DNA and have been confirmed via nucleotide blast (NCBI). Primers for GH mRNA are complementary to the mRNA of GH isoforms 1 and 2, which are the two major isoforms of GH found in humans. Of the other 3 isoforms of GH, isoform 3 is present only in rare cases of GH deficiency [11], and isoforms 4 and 5 appear to arise from post-translational modifications of GH isoforms 1 and 2 (to which the primers are complementary) or to be artifacts produced during purification of GH from pituitary extracts [11]. Primer concentrations were optimized following a published protocol [12]: 100 nmol/L reverse primer (SEQ ID NO:3; BK369) and 300 nmol/L forward primer (SEQ ID NO:2; BK158) concentrations for GH mRNA and 300 nmol/L forward (SEQ ID NO:5; GAPDH368) and reverse (SEQ iD NO:6; GAPDH561) primer concentrations for GAPDH mRNA. The primers are unmodified, were produced by Invitrogen, and were cartridge purified.

### Primer Sequences:

BK158:
SEQ ID NO: 2
ATCCAGGCTTTTTGACAACG (forward primer).
BK369:
SEQ ID NO: 3
GGAGCAGCTCTAGGTTGGATT (reverse primer).
Nest_GH_rev:
SEQ ID NO: 4
TGGAGGGTGTCGGAATAGAC
GAPDH368:
SEQ ID NO: 5
ATGCTGGCGCTGAGTACGTC (forward primer).
GAPDH561:
SEQ ID NO: 6
GGTGCAGGAGGCATTGCTGATG (reverse primer).

Quantification of GH and GAPDH mRNA

For calibration of the assays for GH mRNA we used a synthetic oligonucleotide (GH96), which contains a 3′-phosphate to prevent participation in the PCR reaction, as a calibrator in each run [13]. GH96 was a truncated segment of the GH mRNA sequence that contained 96 nucleotides and the primer binding sites used in GH mRNA amplification. The calibrator was limited to 96 nucleotides to avoid potential limitations of oligonucleotide synthesis. A series of 10-fold serial dilutions of GH96 was used for the calibration curve from which the copy number of unknown samples was calculated. We used pituitary-derived RNA as a quality control material; 300 femtograms (fg) of pituitary-derived RNA is similar in GH mRNA copy number to our volunteer samples. Additionally, we determined relative concentrations of GAPDH mRNA in each sample by assigning a copy number of unity to a Cycle of Quantification (CQ) of 22.76. By utilizing the change in GAPDH CQ between each sample, we determined the relative GAPDH copy number in each sample and divided our absolute GH mRNA copy number by the relative GAPDH copy number to normalize GH mRNA copy number.

PCR Product Characterization

In addition to quantitative analysis of PCR product amplification using SYBR Green I fluorescence, RT-PCR products were characterized by melting curve analysis and by 1.5% agarose gel electrophoresis. To further confirm the identity of the product, the forward and reverse strands of the RT-PCR products were sequenced with Applied Biosystems 3730 DNA Sequencer using the BigDye Terminator v3.1 chemistry.

Assay Reproducibility

To characterize the inter- and intra-assay precision, we performed 10 different nested-primer assays, as described above, over 9 days. We measured GH mRNA in each of 3 samples in each run: 2 whole-blood-derived RNA samples and pituitary-derived RNA (300 fg/assay). Ten-fold dilutions of the synthetic oligonucleotide (GH96), with 90,000 to 9 copies per reaction, were used as calibrators in each run.

qPCR Data Analysis

The qPCR results were analyzed using Roche LightCycler LCDA software (version 3.5.28). The software determined CQ for each sample and the CQ was calculated as 2nd derivative maximum. Significance of differences between patients with acromegaly and control subjects was assessed in two ways: ANOVA of log-transformed data with post-hoc pairwise comparison and Kruskall-Wallis ANOVA based upon the ranked data values. Both ANOVAs were performed by use of SAS version 9.1 software. Graph Pad software was used for calculation of Spearman correlation coefficients, unpaired t-test, linear least squares regression, and receiver operating characteristic analyses.

### Results

### Example 4

### Assay Validation

Representative nested-primer real-time PCR amplifications of GH mRNA are shown in Example 4, FIG. 1A. The amplifications from both whole blood and pituitary produced amplicons with the size (170 bp), melting temperature (85.5° C.) and sequences predicted for products of GH mRNA (See Supplemental Data), with no evidence of amplification of DNA.

The reverse-transcription quantitative nested PCR assay was linear from ≦90 to ≧90,000 copies/reaction of the synthetic oligonucleotide standard (GH96) (Example 4, FIG. 1B). In 10 separate runs on 9 days, the mean slope of the regression line was −3.7 with an SD=0.19 (range: −3.26 to −3.85, r²≧0.99) indicating stability of the calibration and a PCR efficiency of 86% (SD 7%).

GH mRNA was quantifiable by the RT-qPCR assay in all samples of whole blood that were tested. No signal was measurable in samples of plasma or saliva. The between-run imprecision (CV) results for two representative samples of blood RNA analyzed in each of 10 runs were 46% and 31% (at mean concentrations of 2630 and 3400 copies/mL of blood, respectively). The CV of the C_{Q} was 5% at both 90 copies per reaction (similar to blood samples) and 300 fg pituitary RNA per reaction. The C_{Q} of the NTC was not calculated as there was no amplification of non-specific products in the nested PCR assay.

Biological Variation of GH mRNA in Blood

The within-person variation (CV) of GH mRNA over a period of one year (n=17) was 28% (range of concentrations 4170 to 11110 copies/mL blood, Example 4, FIG. 2A). When results were normalized to GAPDH expression, the CV increased to 40%, (range 2500 to 15700 normalized copies/mL blood), presumably reflecting the combined imprecision of two assays (GH and GAPDH mRNAs). The range of copy numbers amongst the control group (males ages 27-36 years old) was 2390 to 5260 copies/mL blood (between-person variability CV of 24%). When results were normalized to GAPDH expression in each sample, the inter-individual variation amongst the control group was similar to the variation assessed without normalization (range 2310 to 7980 normalized copies/mL blood, CV=50%). None of the 17 results in the dataset for the within-person study were included in determining the range of results in the study of normal subjects due to a desire to avoid bias.

The copy number of GH mRNA was not significantly correlated with age of subjects (Spearman r=−0.2925, approximate P=0.15) (data not shown) or with gender (unpaired t-test, P>0.05) (FIG. 2B). We have not examined gender differences within each group statistically because of the small numbers of males and females within each group.

GH mRNA in Patients with Acromegaly

In contrast to the findings of Thakkar et al, the mean GH mRNA in patients with acromegaly in our study did not differ significantly (ANOVAs) from the mean value in control subjects (Example 4, FIG. 3) [7]. Spearman correlation analyses showed no statistically significant correlation of GH mRNA with IGF-I concentration (r=−0.36, P=0.39), further suggesting that GH mRNA in blood is not increased by GH hyper-secretion seen in patients with acromegaly. The mean circulating cellular GH mRNA copy numbers were not statistically different in patients with acromegaly prior to surgery compared with others who had undergone surgery and were still receiving medical therapy for acromegaly (with Octreotide or Pegvisomant), (10410 [range 1080-28970] copies/mL blood and 12200 [range 2280-48150] copies/mL blood, respectively, P=0.9). The group of patients studied before treatment was distinct from the group of patients studied after treatment.

GH mRNA in Patients Receiving rhGH

In individuals receiving physiologic replacement doses of rhGH (0.2-0.8 mg/day) for hypopituitarism, the mean GH mRNA was not different from that in controls: 4690 (range 360 to 8310) copies/mL blood (Example 4, FIG. 3), or 4120 (range 510 to 9250) normalized copies/mL blood when normalized to GAPDH expression (not shown). The GH mRNA showed no statistically significant correlation with the IGF-I concentration (Spearman r=0.34, P=0.33).

Receiver Operating Characteristic (ROC) Analysis of GH mRNA

We performed an ROC analysis of GH mRNA in controls compared with individuals with acromegaly and those receiving therapeutic replacement doses of rhGH as therapy. The area under the curve is 0.65 (95% CI 0.44-0.86), which demonstrates that there is limited discrimination between controls, individuals with acromegaly, and therapeutic rhGH users.

GH mRNA Concentrations Corresponding to WBC Concentrations

B-cells are known to produce GH [14] and thus are the likely source of GH mRNA we are measuring in the assay. Within our data set, the range of white blood cell concentration ranges from 4.42 k/μL to 9.82 k/μL and B-cell concentration ranged from 1.37 k/μL to 1.73 k/μL. There was no significant correlation between GH mRNA copy number and WBC count (Spearman r=0.40, P=0.52).

### Discussion

### Example 4

We have developed a sensitive assay that is capable of quantifying as few as 90 copies of GH mRNA per mL of blood. The assay is specific for GH mRNA as shown by melting curve analysis, agarose gel electrophoresis, and sequencing of amplicons. The concentrations of GH mRNA in blood show only limited variability within a healthy individual over the course of a year, and the between-person variability among young individuals is similar to that seen within a person. This newly designed assay is capable of quantifying GH mRNA at copy numbers over a range ≧10,000 fold.

Contrary to a previous report [7], the mean concentration of GH mRNA was not increased in patients with acromegaly. In the present study the mean copy number in these patients was similar to that in the normal controls with substantial overlap between the two groups (Example 4, FIG. 3). We cannot compare absolute concentrations in the two studies as Thakkar et al reported only raw C_{Q} values as indicators of relative concentration of GH mRNA in blood [7]. The C_{Q} values reported by Thakkar et al were −4.694 and −0.044 for patients and controls which correspond to mean concentrations that were 26 times higher in patients with acromegaly than in controls, assuming 100% efficiency of amplification. In contrast, with the quantitative assay in the present study, no individual patient value was 26 times above the mean of the controls. The reasons for this difference are not clear.

Thakkar and colleagues did not discuss whether the 12 patients with acromegaly that they studied were treated or untreated and such information may be necessary in drawing conclusions about the influence of pituitary status on concentrations of circulating cellular GH mRNA [7]. However, in our study the mean GH mRNA concentrations were similar in untreated and treated acromegaly patients (all of whom were undergoing medical treatment after pituitary surgery). Thus, in untreated patients with acromegaly, the mean GH mRNA copy number was 10410, similar to the mean concentration in treated patients (12200 copies of GH mRNA/mL of blood, P=0.9). Interestingly, Octreotide, a somatostatin analogue with which three of the patients were being treated, did not appear to affect the concentration of GH mRNA in whole-blood. This finding is consistent with that of a prior study which showed that somatostatin had no effect on release of GH from cultured lymphocytes [4].

Our finding of similar GH mRNA concentrations in patients with acromegaly, controls, and in patients receiving GH replacement also contrasts with the report of Hattori et al indicating that exogenous GH increased release of GH from cultured lymphocytes [15]. Our studies, however, measured messenger RNA abundance whereas that of Hattori et al measured release of GH (protein); different results of the two studies would be expected if rhGH altered translation of the message or release of GH rather than gene transcription. Moreover, the doses of rhGH in our study were physiological and should not be expected to produce effects different from the effects of endogenous GH in the healthy volunteers. By contrast, the studies of cultured cells compared conditions with and without high concentrations of GH.

The doses of rhGH administered to hypopituitary patients in this study were physiologic replacement doses that ranged from 0.2 mg/day to 0.8 mg/day and, not surprisingly, did not produce different mRNA concentrations than were present in controls. In contrast, the intention of athletes is to achieve supraphysiologic effects of the hormone. Athletes who dope with rhGH are thought to use doses of GH that are at least ten-fold higher than therapeutic replacement doses. Indeed, in the study to challenge the isoform ratio immunoassay as a technique to detect doping with GH, moderate doses of rhGH (0.033 mg/kg/day) and larger doses (0.083 mg/kg/day) were administered to volunteers [16]. At these doses, a 70 kg individual would receive 2.3 mg/day and 5.8 mg/day. Similarly, the pattern of GH secretion by a pituitary tumor is unlike the daily bolus injections of high doses of rhGH used by athletes. Addressing the question of usefulness of this testing for detection of doping will require careful studies with injections of rhGH in athletes. Such studies are attractive in view of the limited variability of GH mRNA among young individuals and the low within-person variability in a normal individual over time, both of which are valuable attributes of a test for doping.

### Example 4

### Bibliography

1. Muller E E, Locatelli V, Cocchi D. Neuroendocrine control of growth hormone secretion. Physiol Rev 1999; 79:511-607.
2. Chanson P, Salenave S. Acromegaly. Orphanet J Rare Dis 2008; 3:17.
3. Lopes M B. Growth hormone-secreting adenomas: pathology and cell biology. Neurosurg Focus 2010; 29:E2.
4. Hattori N, Ikekubo K, Ishihara T, Moridera K, Hino M, Kurahachi H. Spontaneous growth hormone (GH) secretion by unstimulated human lymphocytes and the effects of GH-releasing hormone and somatostatin. J Clin Endocrinol Metab 1994; 79:1678-1680.
5. Rapaport R, Sills I N, Green L, et al. Detection of human growth hormone receptors on IM-9 cells and peripheral blood mononuclear cell subsets by flow cytometry: correlation with growth hormone-binding protein levels. J Clin Endocrinol Metab 1995; 80:2612-2619.
6. Hattori N. Expression, regulation and biological actions of growth hormone (GH) and ghrelin in the immune system. Growth Horm IGF Res 2009; 19:187-197.
7. Thakkar H, Butt A N, Powrie J, Holt R, Swaminathan R. Circulating nucleic acids in the assessment of endogenous growth hormone production Ann N Y Acad Sci 2008; 1137:58-65.
8. Kooijman R, Willems M, De Haas C J, et al. Expression of type I insulin-like growth factor receptors on human peripheral blood mononuclear cells. Endocrinology 1992; 131:2244-2250.
9. Short PAXGene Protocol.
10. Bustin S A, Benes V, Garson J A, et al. The MIQE guidelines: minimum information for publication of quantitative real-time PCR experiments. Clin Chem 2009; 55:611-622.
11. Baumann G P. Growth hormone isoforms. Growth Horm IGF Res 2009; 19:333-340.
12. Nolan T, Hands R E, Bustin S A. Quantification of mRNA using real-time RT-PCR. Nat Protoc 2006; 1:1559-1582.
13. Vermeulen J, Pattyn F, De Preter K, et al. External oligonucleotide standards enable cross laboratory comparison and exchange of real-time quantitative PCR data. Nucleic Acids Res 2009; 37:e138.
14. Hattori N, Saito T, Yagyu T, Jiang B-H, Kitagawa K, Inagaki C. GH, GH Receptor, GH Secretagogue Receptor, and Ghrelin Expression in Human T Cells, B Cells, and Neutrophils. J Clin Endocrinol Metab 2001; 86:4284-4291.
15. Hattori N, Shimomura K, Ishihara T, et al. Growth hormone (GH) secretion from human lymphocytes is up-regulated by GH, but not affected by insulin-like growth factor-I. J Clin Endocrinol Metab 1993; 76:937-939.
16. Bidlingmaier M, Suhr J, Ernst A, et al. High-Sensitivity Chemiluminescence Immunoassays for Detection of Growth Hormone Doping in Sports. Clin Chem 2009; 55:445-453.

The disclosures of each and every patent, patent application, and publication cited herein are hereby incorporated by reference herein in their entirety.

Headings are included herein for reference and to aid in locating certain sections. These headings are not intended to limit the scope of the concepts described therein, and these concepts may have applicability in other sections throughout the entire specification.

While this invention has been disclosed with reference to specific embodiments, it is apparent that other embodiments and variations of this invention may be devised by others skilled in the art without departing from the true spirit and scope of the invention.

Other methods which were used but not described herein are well known and within the competence of one of ordinary skill in the art of cell biology, molecular biology, and clinical medicine. The invention should not be construed to be limited solely to the assays and methods described herein, but should be construed to include other methods and assays as well. One of skill in the art will know that other assays and methods are available to perform the procedures described herein.

What is claimed is:

1. A method for detecting a change in the amount of at least one microRNA (miRNA) associated with administration of growth hormone to a subject, said method comprising measuring the amount of said at least one miRNA in a sample obtained from said subject, wherein an increase or decrease in said miRNA level compared to the amount in an otherwise identical sample where no growth hormone had been administered is an indication that growth hormone or a biologically active fragment, homolog, or derivative thereof, or an agonist of growth hormone or growth hormone signaling pathways, was administered to said subject.

2. The method of claim 1, wherein said sample is a blood sample.

3. The method of claim 1, wherein said at least one miRNA increases after administration of said growth hormone or a biologically active fragment, homolog, or derivative thereof to said subject.

4. The method of claim 3, wherein said at least one miRNA is selected from the group consisting of hsa-miR-510, hsa-miR-200b*, and hsa-miR-1272.

5. The method of claim 1, wherein said at least one miRNA decreases after administration of said growth hormone, or a biologically active fragment, homolog, or derivative thereof to said subject.

6. The method of claim 5, wherein said at least one miRNA is selected from the group consisting of miR-663, miR-2861, miR-3185, and miR-3152.

7. The method of claim 1, wherein the amounts of at least two of said miRNAs decrease.

8. The method of claim 7, wherein the amounts of at least three of said miRNAs decrease.

9. The method of claim 6, wherein the amounts of each of said miR-663, miR-2861, miR-3185, and miR-3152 decrease.

10. The method of claim 1, wherein said growth hormone is recombinant growth hormone.

11. The method of claim 1, wherein said growth hormone is human growth hormone.

12. The method of claim 1, wherein said subject is human.

13. The method of claim 1, wherein said subject is an athlete.

14. The method of claim 1, wherein samples are obtained at different times from said subject to monitor the effectiveness of growth hormone therapy when said subject is undergoing replacement therapy or to monitor the amounts when said subject is an athlete.

15. The method of claim 14, wherein said subject said growth hormone has the sequence of SEQ ID NO:1.

16. The method of claim 1, wherein said subject is being treated for a disease or disorder associated with aberrant levels of growth hormone.

17. A method for measuring the amount of growth hormone mRNA in a sample, said method comprising:
a) purifying RNA from said sample;
b) subjecting said purified RNA to reverse transcription and a first round of PCR using forward and reverse primers for growth hormone mRNA, obtaining PCR products, and quantifying said PCR products; and
c) subjecting said products of said first round of PCR to a second round of PCR, wherein the forward primer used is the same as in step b, but the reverse primer is a different nested reverse primer than the reverse primer used in step b, further wherein said first and second round primers are exon-exon junction primers, and quantifying the products of said second round of PCR, thereby measuring the amount of growth hormone mRNA in a sample.

18. The method of claim 17, wherein said sample is a blood sample.

19. The method of claim 17, wherein said sample is a human sample.

20. The method of claim 17, wherein said reverse primer for nested PCR of step c has the sequence of SEQ ID NO:4.

21. The method of claim 17, wherein said forward primer has the sequence of SEQ ID NO:2.

22. The method of claim 17, wherein No Template Controls are used for comparison to said sample.

23. The method of claim 17, wherein a proteinase K digestion is performed and said sample is vortexed for about 15 seconds during step a.

24. The method of claim 17, wherein said first round of PCR comprises at least about 25 cycles of PCR and said second round of PCR comprises at least about 25 cycles of PCR.

25. The method of claim 17, wherein said primers are complementary to mRNA of growth hormone isoforms 1 and 2.

26. The method of claim 17, wherein said PCR products are characterized by at least one of melting curve analysis, agarose gel electrophoresis, and sequencing.

27. The method of claim 17, wherein said method comprises the use of nested PCR and growth hormone mRNA amplification.

28. The method of claim 17, wherein amplifications produce amplicons of 170 bp, with melting temperatures of 85.5° C., and with the predicted sequence of growth hormone mRNA.

29. The method of claim 17, wherein a reverse-transcription quantitative nested PCR assay of said method is linear from about 90 to about 90,000 copies/reaction of a synthetic oligonucleotide standard.

30. The method of claim 17, wherein the amount of growth hormone mRNA in a sample from a subject with acromegaly prior to surgery or treatment ranges from about 1,000 copies/mL blood to about 30,000 copies/mL blood.

31. The method of claim 17, wherein the amount of growth hormone mRNA in a sample from a subject with acromegaly after surgery but still receiving treatment ranges from about 2,200 copies/mL blood to about 50,000 copies/mL blood.

32. The method of claim 17, wherein the amount of growth hormone mRNA in a sample from a subject receiving growth hormone replacement therapy for hypopituitarism is from about 350 copies/ml blood to about 9,500 copies/ml blood.

33. The method of claim 17, wherein said growth hormone mRNA is derived from B cells in blood.

34. The method of claim 17, wherein said method can quantify as few as about 90 copies of growth hormone mRNA per mL of blood.


SEQUENCE LISTING (7 sequences)

SEQ ID NO: 1 - protein, 217 aa, Homo sapiens
MATGSRTSLLLAFGLLCLPWLQEGSAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 2 - DNA, 20 bp, homo sapiens
atccaggctttttgacaacg

SEQ ID NO: 3 - DNA, 21 bp, Homo sapiens
ggagcagctctaggttggatt

SEQ ID NO: 4 - DNA, 20 bp, Homo sapiens
tggagggtgtcggaatagac

SEQ ID NO: 5 - DNA, 20 bp, Homo sapiens
atgctggcgctgagtacgtc

SEQ ID NO: 6 - DNA, 22 bp, Homo sapiens
ggtgcaggaggcattgctgatg

SEQ ID NO: 7 - DNA, 822 bp, Homo sapiens
aggatcccaaggcccaactccccgaaccactcagggtcctgtggacagctcacctagctgcaatggctacaggctcccggacgtccctgctcctggcttttggcctgctctgcctgccctggcttcaagagggcagtgccttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagccttgtcctaataaaattaagttgcatca